SEQUENCE LISTING (2 sequences)

SEQ ID NO: 1 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 2 - protein, 609 aa, Homo sapiens [sequence omitted]

(19) United States
(12) Patent Application Publication
(10) Pub. No.: US 2016/0045606 A1
(43) Pub. Date: Feb. 18, 2016

(54) PARTIALLY-DENATURED PROTEIN HYDROGELS

(71) Applicant: Northwestern University, Evanston, IL (US)

(72) Inventors: Kevin Baler, Chicago, IL (US); Igal G. Szleifer, Evanston, IL (US); Guillermo A. Ameer, Chicago, IL (US)

(21) Appl. No.: 14/829,186

(22) Filed: Aug. 18, 2015

Related U.S. Application Data
(60) Provisional application No. 62/038,614, filed on Aug. 18, 2014.

(51) Int. Cl. A61K 47/42; A61K 47/48

(52) U.S. Cl. CPC A61K 47/42; A61K 47/48784

(57) ABSTRACT

Provided herein are partially-denatured protein (e.g., albumin) hydrogels and methods of manufacture (e.g., pH induction) and use (e.g., drug delivery) thereof.

### CROSS REFERENCE TO RELATED APPLICATIONS

The present application claims the benefit of U.S. Provisional Patent Application Ser. No. 62/038,614, filed Aug. 18, 2014, which is incorporated by reference in its entirety.

### FIELD

Provided herein are partially-denatured protein (e.g., albumin) hydrogels and methods of manufacture (e.g., pH induction) and use (e.g., drug delivery) thereof.

### BACKGROUND

Serum albumin is widely used clinically as a critical component for use in solubilizing diagnostic and therapeutic products due to its versatility as a drug carrier (Peters T. All About Albumin: Elsevier; 1995; herein incorporated by reference in its entirety). Concurrently, biological hydrogels are extensively used in medical applications due to their fundamental biocompatibility and intrinsic similarities to the extracellular matrix of certain tissues (Peppas et al. Adv Mater. 2006; 18(11):1345-60; Slaughter et al. Adv Mater. 2009; 21(32-33):3307-29; herein incorporated by reference in their entireties). Hydrogels have been synthesized from a variety of biomacromolecules, such as serum albumin, by forming intermolecular cross-links via thermal or chemical methods (Peppas et al. Adv Mater. 2006; 18(11):1345-60; Slaughter et al. Adv Mater. 2009; 21(32-33):3307-29; herein incorporated by reference in their entireties).

### SUMMARY

Provided herein are partially-denatured protein (e.g., albumin) hydrogels and methods of manufacture (e.g., pH induction) and use (e.g., drug delivery) thereof. In some embodiments, the partially-denaturated proteins form protein aggregates, but maintain secondary structure (e.g., partial secondary structure). In some embodiments, the partially-denatured proteins (e.g., albumin) self-assemble into a hydrogel. In some embodiments, the partially-denatured proteins (e.g., albumin) maintain ligand binding functionality within the hydrogel. In some embodiments, partially-denatured proteins (e.g., partially-denatured albumin (e.g., BSA, HSA, etc.)) are electrostatically-denatured proteins.

In some embodiments, provided herein are compositions comprising a hydrogel of partially-denatured albumin. In some embodiments, the partially-denatured albumin is electrostatically denatured (a structurally distinct entity from that produced by thermal denaturation). In some embodiments, the partially-denatured albumin retains a portion of the secondary structure of albumin, but has altered tertiary structure. In some embodiments, the partially-denatured albumin is F-form albumin. In some embodiments, the partially-denatured albumin in the hydrogel retains the drug-binding functionality of undenatured albumin. In some embodiments, the partially-denatured albumin in the hydrogel binds all-trans retinoic acid. In some embodiments, the albumin has at least 70% sequence identity with SEQ ID NO: 1. In some embodiments, the albumin has at least 70% sequence identity with SEQ ID NO: 2. In some embodiments, the albumin in the hydrogel is not crosslinked. In some embodiments, the albumin in the hydrogel is substantially not crosslinked (e.g., >99% of residues in a sample comprising a population of albumin proteins are not crosslinked (e.g., >99.9%, >99.99%, etc.). In some embodiments, the albumin in the hydrogel is not completely denatured. In some embodiments, the hydrogel is non-toxic to humans (e.g., biocompatible).

In some embodiments, provided herein are methods of producing a partially-denatured albumin hydrogel comprising exposing albumin to aqueous conditions under pH 5.0 (e.g., <4.5, <4.0, <3.6, etc.). In some embodiments, methods further comprise a step of returning the partially-denatured albumin hydrogel to above pH 5.0 (e.g., >5.0, >5.5, >6.0, >6.5, >7.0 etc.).

In some embodiments, provided herein are compositions comprising: (i) a hydrogel of partially-denatured albumin, and (ii) cells or one or more active agents. In some embodiments, the cells or one or more active agents are embedded within the hydrogel. In some embodiments, the cells or one or more active agents are in a reservoir within the hydrogel. In some embodiments, the hydrogel has a diffusion coefficient for the active agent between 1×10⁻⁷ and 1×10⁻⁴ mm²/minute.

In some embodiments, provided herein are devices comprising at least one base material, wherein at least a portion of the at least one base material is coated in a hydrogel of partially-denatured albumin. In some embodiments, the device is a medical device, a surgical device, or an implantable device. In some embodiments, the base material is biocompatible. In some embodiments, the base material is inert. In some embodiments, the hydrogel composition further comprises an active agent.

In some embodiments, provided herein are drug-delivery devices comprising a hydrogel of partially-denatured albumin. In some embodiments, the hydrogel further comprises an active agent.

In some embodiments, provided herein are methods of treating or preventing a condition in a subject comprising: (a) administering a drug-delivery device comprising a hydrogel of partially-denatured albumin and a therapeutic agent to the subject, wherein the therapeutic agent is effective in the treatment or prevention of the condition; and (b) allowing the therapeutic agent to elute from the hydrogel. In some embodiments, provided herein are methods of treating or preventing a condition in a subject comprising: (a) administering a drug-delivery device comprising a hydrogel of partially-denatured albumin and a therapeutic agent to the subject, wherein the therapeutic agent is effective in the treatment or prevention of the condition; and (b) allowing the hydrogel to degrade, thereby releasing the therapeutic agent. In some embodiments, the hydrogel degrades by enzymatic degradation.

In some embodiments, provided herein are pharmaceutical preparations comprising a hydrogel of partially-denatured albumin and one or more therapeutic agents. In some embodiments, the therapeutic agent is impregnated within the hydrogel. In some embodiments, the hydrogel is a coating on the exterior of the pharmaceutical preparation. In some embodiments, the hydrogel comprises the internal core of the pharmaceutical preparation. In some embodiments, the therapeutic agent is contained within a reservoir within the hydrogel. In some embodiments, the hydrogel is encapsulated within a film, membrane, shell, or coating.

In some embodiments, provided herein are methods of treating or preventing a condition in a subject comprising: (a) administering the pharmaceutical preparation comprising a hydrogel of partially-denatured albumin and one or more therapeutic agents to the subject, wherein the therapeutic agent is effective in the treatment or prevention of the condition; and (b) allowing the therapeutic agent to elute from the hydrogel.

In some embodiments, provided herein are methods of treating or preventing a condition in a subject comprising: (a) administering the pharmaceutical preparation comprising a hydrogel of partially-denatured albumin and one or more therapeutic agents to the subject, wherein the therapeutic agent is effective in the treatment or prevention of the condition; and (b) allowing the hydrogel to degrade, thereby releasing the therapeutic agent. In some embodiments, the hydrogel degrades by enzymatic degradation.

In some embodiments, provided herein are methods of administering an active agent to a subject comprising: (a) administering composition comprising a hydrogel of partially-denatured albumin and an active agent to the subject; and (b) allowing the active agent to elute from the hydrogel. In some embodiments, the composition is an implantable device, a coating on a device, or a pharmaceutical preparation.

In some embodiments, provided herein are methods of administering an active agent to a subject comprising: (a) administering a composition comprising a hydrogel of partially-denatured albumin and an active agent to the subject; and (b) allowing the hydrogel to degrade, thereby releasing the therapeutic agent. In some embodiments, the composition is an implantable device, a coating on a device, or a pharmaceutical preparation. In some embodiments, the hydrogel degrades by enzymatic degradation.

In some embodiments, provided herein are hydrogel particles comprising partially-denatured albumin hydrogel. In some embodiments, the particle is a microparticle or a nanoparticle. In some embodiments, provided herein is a population of uniformly sized and/or shaped hydrogel particles. In some embodiments, provided herein are methods of producing a hydrogel particle comprising aerosolizing a solution comprising albumin into a solution below pH 5.0.

In some embodiments, hydrogel particles further comprise an active agent. In some embodiments, provided herein are methods of producing a hydrogel particle comprising an active agent, comprising aerosolizing a solution comprising albumin into a solution below pH 5.0, thereby forming the hydrogel particles, and subsequently adding the active agent to the hydrogel particles. In some embodiments, provided herein are methods of producing a hydrogel particle comprising an active agent, comprising adding the active agent to a solution comprising albumin, aerosolizing the solution comprising albumin into a solution below pH 5.0, thereby forming the hydrogel particles.

In some embodiments, provided herein are composite materials comprising: (i) a partially-denatured albumin hydrogel, and (ii) one or more additional materials. In some embodiments, the one or more additional materials are selected from other hydrogels, polymers, elastomers, bioceramics.

In some embodiments, provided herein are biomedical materials comprising partially-denatured protein (e.g., albumin) hydrogels. In some embodiments, the biomedical material is used as a wound dressing, a haemostatic material, or a drug delivery system.

### BRIEF DESCRIPTION OF THE DRAWINGS

FIGS. 1A and B show determination of ionization state of titratable residues for simulations. (A) Chemical structures of titratable residues (ASP, GLU, HIS, LYS, ARG) and the resulting ionized structure at pH 3.5. (B) Localization of ionized residues on albumin at pH 7.4 and pH 3.5. Inset shows the total charge per residue type and for the protein overall at the two pH values. At pH 7.4, the protein has a total charge of −9, while at pH 3.5 the charge is +100 (including the amine terminal group).

FIGS. 2A-C show partial unfolding simulations of albumin with titratable residues set to pH 3.5 ionization states. Domains 1, 2, and 3 are indicated. (A) Snapshots of albumin conformations simulation during partial electrostatically triggered denaturation. (B) Final simulation conformations of albumin at pH 3.5. Locations of positive charges and counterions are represented on the right. (C) Distance measured between the center of mass of domain 1 and domain 3 during simulations with counterions in comparison with physiological albumin at pH 7.4 (blue). Insets depict albumin final conformations along each path.

FIGS. 3A and B show: (A) percentage of helices in each domain for both N and F BSA isoforms. All F domains loose a fraction of their helical content to turn/coil structures during the partial denaturation in comparison to N conformations; and (B) circular dichroism data of dilute solutions of BSA (0.005 wt %) at low pH and high temperature showing the relative degree of secondary structure denaturation. Electrostatically triggered denaturation avoids total loss of secondary structures as observed in thermal denaturation.

FIG. 4 shows solvent accessible surface areas for hydrophobic and hydrophilic moieties in the N and F BSA isoforms represented for (a) each domain individually and (b) the change due to the N—F transition. The SAS increases for hydrophobic moieties and decreases hydrophilic ones in the F isoform.

FIG. 5 shows the solvent accessible surface areas for ASP and GLU residues in the N and F BSA isoforms in the total protein and in for each domain. Normally hydrophilic residues ASP and GLU face the solvent at pH 7.4 but are hydrophobic when protonated at pH 3.5.

FIG. 6 shows dimerization of two F conformation albumin proteins. (a) Minimum distance measured between two F-isoform BSA structures placed near each other and simulated with and without system neutralizing counterions. Proteins with counterions allowed proteins to stay within 0.25 nm of each other until they separated after 36 ns. Absence of counterions allowed unscreened repulsive electrostatic interactions to rapidly overcome attractions. (b) Configurations of two proteins from (a) at 10 ns. The top pair corresponds to the no counterion simulation and the bottom pair corresponds to the counterion simulation.

FIGS. 7A-E show explicit counterion and Poisson-Boltzmann (PB) calculated electrostatic surface potentials for (A) single proteins and (B) aggregated proteins. Explicit counterion calculations result in a more negative electrostatic potential when compared to PB. Explicit calculations that ignore counterion contributions duplicate the positive electrostatic potentials shown by PB. Potentials are shown at the Connolly and SAS surfaces are shown for all cases. For clarity, aggregated proteins are shaded individually to help differentiate them in the potential surface representation. (C) Residue ARG 208 is an example of a residue which is has a positive potential when calculated with PB and a negative potential when calculated with explicit counterions. A histogram of the distances to the nearest Cl⁻ ion for the charged N⁺ atom on ARG 208 demonstrates that this residue is typically bound to Cl⁻. (D) Localization of residues selected for further analysis. Inset depicts ARG 484, LYS 474, GLU 478, and LYS 350 are all located near the point of contact between the two proteins. (E) Histograms of distances to nearest Cl ions for the four selected residues near the point of contact in d and magnified representations of charged atoms associated with Cl⁻.

FIGS. 8A-C shows formation of albumin hydrogels by electrostatically triggered partial protein denaturation. (A) Ribbon diagrams depicting the partial denaturation of N-form to F-form albumin leading to protein-protein aggregation and eventual hydrogel formation. Inverted vial depicts 20 wt % PBSA formed at 25° C. Tubular PBSA cylinder made in mold at 37° C. Cryo-SEM images of freeze-fractured hydrogels formed by electrostatic triggering method at pH 3.5 at 37° C. (B) or by thermal denaturation at 80° C. method (C).

FIGS. 9A-F show graphs depicting the mechanical properties of BSA hydrogels demonstrating PBSA hydrogels are softer than TBSA hydrogels. (A), Young's modulus of BSA solutions (17 wt % BSA) with different pH values ranging from 2.5-6 incubated at 37° C. (n=4 for each pH value). BSA forms (E, F, N) are mapped below the plot according to their pH transition values. (B), Young's modulus of 20 wt % PBSA (pH 3.5 incubated at 37° C., n=4) and 20 wt % TBSA (pH 7.4 incubated at 80° C., n=8) hydrogels measured by mechanical indentation. Error bars in (A) and (B) represent the standard deviation of the data set. (C), Rheological characterization of PBSA formation kinetics for protein concentration 20, 18, and 16 wt %. (D), Crossover points between G′ and G″ for each concentration of PBSA at 20, 18, and 16 wt %. (E), Rheological characterization of TBSA formation kinetics for protein concentration 20, 18, 16, 14, and 12 wt %. (F) Young's modulus of PBSA (pH 3.5 incubated at 37° C., n=4) and TBSA (pH 7.4 incubated at 80° C., n=8) hydrogels with increasing BSA concentration measured by mechanical indentation.

FIG. 10 shows the effect of various chemical environments on PBSA hydrogel integrity. Small cylindrical PBSA hydrogels (0.5 cm diameter), acid-leached in DMEM for 3 days until the pH returned to neutral pH, were placed in different solutions and photographed over the course of three months. These images are representative of the larger sample set (n=4) and demonstrate hydrogel degradation resistance to acid, base, and salt conditions. Urea and 10% SDS degrade the gels within 17 hrs while disulphide bond reduction by β-ME results in hydrogel swelling.

FIG. 11 shows histological images demonstrating the biological response to subcutaneously implanted PBSA and TBSA hydrogels. Tissues with implanted PBSA gel disks (a, e), TBSA gel disks (b, f), pH 7.4 BSA solution injection (c, g), and saline control injections (d, h) at 4 days (a, b, c, d) and 4 weeks (e, f, g, h). Samples were stained with H&E. Histological images are aligned to each other at the original interface between the implant and the tissue lining the subcutaneous pocket. Images for each time point are from the same rat and are representative of the group population overall.

FIGS. 12A-D shows (A) atRA binding to N-form albumin and F-form albumin measured via fluorescence quenching of tryptophan residue TRP 213 at 340 nm; (B) Fluorescence intensity for N isoform BSA and F isoform BSA is consistent before addition of atRA. Addition of increasing molar concentration of atRA quenches both N378 form albumin and F-form albumin fluorescence intensity; (C) A greater fraction of initial fluorescence is quenched in N-form albumin than in F-form albumin indicating altered binding affinity; and (D) Release of atRA into PBS at 37° C. from F-form albumin and TBSA hydrogels.

FIG. 13 shows localization of 10 atRA molecules binding to F-form albumin and N-form albumin after fully atomistic MD simulations for 100 ns. Top panel depicts F-form albumin binding sites located primarily in domain I and II. Clusters of atRA also formed aggregates on the protein surface. Bottom panel depicts N-form albumin binding sites located in all three domains.

FIGS. 14A and B show separation distance between individual atRA molecules and their nearest protein surface residues during 100 ns MD simulations for both F-form albumin (A) and N-form albumin (B). Each individual atRA becomes bound to the protein surface when the separation distance drops below 0.14 nm. atRA molecules that bind to the surface become effectively immobilized within 100 ns.

FIGS. 15A and B show potential of Mean Force calculations from umbrella sampling simulations for atRA molecules entering Site 1 on both F-form albumin and N-form albumin. In this particular site, the ΔG=−41 kJ/mol for Site 1 on F-form albumin (A) and ΔG=−13 kJ/mol for Site 1 on N-form albumin (B).

FIG. 16 shows atRA released from PBSA and TBSA hydrogels remains bioactive and reduces the migration of smooth muscle cells as evaluated by a 24 hr scratch wound assay. All cultures are serum starved to limit proliferation. Compared to controls, all cells exposed to atRA (direct atRA, eluted from PBSA, and eluted from TBSA) exhibited a significant (p<0.05) reduction in cell migration. Scale bar=100 μm.

FIG. 17 shows an exemplary PBSA microparticle fabrication setup (left) and microparticles generated thereby (right).

### DEFINITIONS

As used herein, the term “hydrogel” refers to a class of polymeric materials which swell in aqueous medium, but which do not dissolve in water.

As used herein, the term “particle” refers to solid, semisolid, or gelatinous masses that may be spherical, elongate, rod, or irregularly shaped. Particles may be homogeneous in composition, may comprise an outer shell or membrane and an inner core, or may be of any other suitable composition.

As used herein, the term “microparticles” refers to particles having a mean diameter of greater than 1 μm but less than 1 mm. Microparticles may be spherical or of another suitable shape (e.g., oblong, rod-like, etc.).

As used herein, the term “microspheres” refers to approximately-spherical particles having a mean diameter of greater than 1 μm but less than 1 mm. Particles that are “approximately” spherical have diameters in all dimensions within 10% of the mean diameter (e.g., mean diameter of 50 μm, x-diameter of 55 μm, y-diameter of 52 μm, and z-diameter of 45 μm).

As used herein, the term “nanoparticles” refers to particles having a mean diameter of greater than 1 nm but less than 1 μm. Nanoparticles may be spherical or of another suitable shape (e.g., oblong, rod-like, etc.).

As used herein, the term “nanospheres” refers to approximately-spherical particles having a mean diameter of greater than 1 nm but less than 1 μm. Particles that are “approximately” spherical have diameters in all dimensions within 10% of the mean diameter (e.g., mean diameter of 100 nm, x-diameter of 105 nm, y-diameter of 110 nm, and z-diameter of 90 nm).

As used herein, term “homogeneous population” refers to a group of objects (e.g., microparticles, nanoparticles, etc.) wherein at least 80% of the objects in the group are of the same type (e.g., same chemical and structural composition, same shape, same size, etc.), within 10% error. In some exemplary embodiments a population is “X % homogeneous” when at least about X % of the objects in the population are of the same type, within 10% error. For example, a population of partially-denatured albumin microspheres in which 90% of the microspheres in the population have a diameter of within 10% of 100 μm is 90% homogeneous.

As used herein, the term “active agent” includes an agent, drug, compound, composition of matter or mixture thereof which provides some diagnostic, prophylactic, or pharmacologic, often beneficial, effect. This includes foods, food supplements, nutrients, drugs, vaccines, vitamins, and other beneficial agents. As used herein, the terms further include any physiologically or pharmacologically active substance that produces a localized or systemic effect in a patient. Active agents include antibiotics, antibodies, antiviral agents, anepileptics, analgesics, anti-inflammatory agents and bronchodilators, and viruses and may be inorganic and organic compounds, including, without limitation, drugs which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, autacoid systems, the alimentary and excretory systems, the histamine system and the central nervous system. Suitable agents may be selected from, for example, polysaccharides, steroids, hypnotics and sedatives, psychic energizers, tranquilizers, anticonvulsants, muscle relaxants, antiparkinson agents, analgesics, anti-inflammatories, muscle contractants, antimicrobials, antimalarials, hormonal agents including contraceptives, sympathomimetics, polypeptides, and proteins capable of eliciting physiological effects, diuretics, lipid regulating agents, antiandrogenic agents, antiparasitics, neoplastics, antineoplastics, hypoglycemics, nutritional agents and supplements, growth supplements, fats, antienteritis agents, electrolytes, vaccines and diagnostic agents.

As used herein, the term “biodegradable” refers to materials that degrade over time upon exposure to physiologic conditions (e.g., by enzymatic action, by hydrolytic action and/or by other similar mechanisms).

As used herein, the term “bioabsorbable” refers to materials that dissipate upon exposure to physiologic condition (e.g., within tissue, within a body, etc.), independent of which mechanisms by which dissipation can occur, such as dissolution, degradation, absorption and excretion.

As used herein the terms “non-bioabsorbable,” “non-biodegradable,” and “biostable” refer to a materials which remains in a tissue or body without substantial degradation or absorption over time (e.g., 1 day . . . 1 week . . . 1 month . . . 1 year, or more).

### DETAILED DESCRIPTION

Provided herein are partially-denatured protein (e.g., albumin) hydrogels and methods of manufacture (e.g., pH induction) and use (e.g., drug delivery) thereof.

### I. Introduction

Experiments were conducted during development of embodiments of the present invention that demonstrate and characterize the use of partially denaturated proteins to form protein aggregates that maintain some degree of secondary structure and self-assemble into a hydrogel that maintains the ligand binding functionality of the protein precursor. In some embodiments, the partial denaturation is induced by changes in pH, rather than commonly used method of thermal denaturation to form hydrogels. In some embodiments, the charging of the amino acids (e.g., by altering pH) is a driving force in this system. Experiments were conducted during development of embodiments of the present invention that demonstrate the formation of nanoparticles/microparticles of partially-denatured protein hydrogels. In some embodiments, the formation of hydrogels by the methods described herein is irreversible or not reversed by exposure to neutral and/or physiological conditions. In some embodiments, the hydrogels are biodegradeable (e.g., in vivo). In some embodiments, the hydrogels retain significant binding affinity to therapeutics despite partial denaturation of the proteins.

### II. Partial Denaturation

In some embodiments, provided herein are hydrogels comprised of aggregates of partially denatured protein. In some embodiments, partial denaturation (e.g., electrostatically induced (e.g., via low pH)) results in solvent exposure of hydrophobic regions of the protein. In some embodiments, the proteins with exposed hydrophobic regions aggregate due to the favorable hydrophobic interactions between the proteins. In some embodiments, partial denaturation is electrostatically induced (e.g., by exposure of the protein to low pH). Electrostatically-induced partial denaturation produces a protein structure (e.g., tertiary structure, quaternary structure, etc.) that is distinct from the protein structure of a protein exposed to thermally-induced partial denaturation.

In some embodiments, partial denaturation (e.g., pH induced) of albumin (e.g., BSA, HSA, etc.) produces a transition from the N-form to the F-form. In certain embodiments, hydrophobic interactions between F-form albumins results in protein aggregation and hydrogel formation. It is contemplated that partial denaturation of other proteins (e.g., proteins with interdomain hydrophobic interactions) produces similar restructuring, aggregation, and hydrogel formation.

### III. Albumin

In some embodiments, the partially-denatured protein (e.g., in a hydrogel) is albumen. In some embodiments, provided herein is partially-denatured (e.g., electrostatically-induced) human serum albumin (HSA; SEQ ID NO: 2). In some embodiments, the partially-denatured (e.g., electrostatically-induced) protein is a fragment of HSA (e.g., 20 amino acids, 30 amino acids, 40 amino acids, 50 amino acids, 60 amino acids, 70 amino acids, 80 amino acids, 90 amino acids, 100 amino acids, 120 amino acids, 140 amino acids, 160 amino acids, 180 amino acids, 200 amino acids, 240 amino acids, 280 amino acids, 320 amino acids, 350 amino acids, 400 amino acids, 450 amino acids, 500 amino acids, 550 amino acids, 600 amino acids, or any ranges therebetween (e.g., 100-200 amino acids, 240-600 amino acids, etc.), etc.). In some embodiments, the partially-denatured (e.g., electrostatically-induced) protein comprises at least 50% sequence identity to all or a portion of HSA (e.g., >50%, >60%, >70%, >75%, >80%, >85%, >90%, >95%, >99%). In some embodiments, provided herein is partially-denatured (e.g., electrostatically-induced) bovine serum albumin (BSA; SEQ ID NO: 1). In some embodiments, the partially-denatured (e.g., electrostatically-induced) protein is a fragment of BSA (e.g., 20 amino acids, 30 amino acids, 40 amino acids, 50 amino acids, 60 amino acids, 70 amino acids, 80 amino acids, 90 amino acids, 100 amino acids, 120 amino acids, 140 amino acids, 160 amino acids, 180 amino acids, 200 amino acids, 240 amino acids, 280 amino acids, 320 amino acids, 350 amino acids, 400 amino acids, 450 amino acids, 500 amino acids, 550 amino acids, 600 amino acids, or any ranges therebetween (e.g., 100-200 amino acids, 240-600 amino acids, etc.), etc.). In some embodiments, the partially-denatured (e.g., electrostatically-induced) protein comprises at least 50% sequence identity to all or a portion of BSA (e.g., >50% . . . >60% . . . >70% . . . >75% . . . >80% . . . >85% . . . >90% . . . >95% . . . >99%). The present invention is not limited to these variants of albumin, and other albumins (e.g., other serum albumins, storage protein ovalbumin, etc.). In some embodiments, a hydrogel contains two or more types of albumin or variations (e.g., fragments) of a single albumin (e.g., HSA).

In some embodiments, albumin is an effective drug carrier (e.g., due to its drug binding/releasing characteristics). In particular, albumin is used as a carrier for molecules (e.g., therapeutics) with low water solubility (e.g., proteins, peptides, hormones, steroids, etc.). In some embodiments, otherwise insoluble or low-solubility active agents are solubilized (e.g., without the use of organic solvents) by interacting (e.g., binding) with albumin. Likewise, albumin is utilized as a carrier and shield for drugs which can produce undesirable side effect during delivery (e.g., parenteral delivery). In some embodiments, the partially-denatured (e.g., electrostatically induced) albumin (e.g., HSA) retains all or a portion of the drug binding capability of the undenatured albumin (e.g., >10% . . . >25% . . . >50% . . . >60% . . . 70% . . . >75% . . . >80% . . . >85% . . . >90% . . . >95% . . . >99%). In some embodiments, the partially-denatured (e.g., electrostatically induced) albumin (e.g., HSA) effectively binds to a drug. In some embodiments, the partially-denatured (e.g., electrostatically induced) albumin (e.g., HSA) has a Kd for an active agent (e.g., drug) that is less than 10⁻⁴ (e.g., <10⁻⁴ . . . <10⁻⁵ . . . <10⁻⁶ . . . <10⁻⁷ . . . <10⁻⁸, or less). In some embodiments, the partially-denatured (e.g., electrostatically induced) albumin (e.g., HSA) has a Kd for an active agent (e.g., drug) that is between 10⁻⁴ and 10⁻¹⁰ (e.g., 10⁻⁴-10⁻⁶, 10⁻⁵-10⁻⁷, 10⁻⁶-10⁻⁸, 10⁻⁷-10⁻⁹, 10⁻⁸-10⁻¹°, 10⁻⁴-10⁻⁷, 10⁻⁵-10⁻⁸, 10⁻⁶-10⁻⁹, 10⁻⁷-10, 10⁻⁴-10⁻⁵, 10⁻⁵-10⁻⁶, 10⁻⁶-10⁻⁷, 10⁻⁷-10⁻⁸, 10⁻⁸-10⁻⁹, 10⁻⁹-10⁻¹⁰, ranges therein, or combinations of such ranges). In some embodiments, the partially-denatured (e.g., electrostatically induced) albumin (e.g., HSA) releases an active agent (e.g., drug) at a rate suitable for therapeutic administration.

### IV. Hydrogels

In some embodiments, because the partially-denatured (e.g., electrostatically induced) albumin (e.g., HSA) forms a hydrogel, delivery of an active agent to a target is no only limited by release from the partially-denatured albumin; rather, the active agent must also diffuse through the hydrogel. In some embodiments, hydrogels of the partially-denatured (e.g., electrostatically induced) albumin (e.g., HSA) have diffusion coefficients between 10⁻³ mm²/minute and 10⁻⁷ mm²/minute (e.g., 10⁻³-10⁻⁴, 10⁻⁴-10⁻⁵, 10⁻⁵-10⁻⁶, 10⁻⁶-10⁻⁷, 10⁻³-10⁻⁵, 10⁻⁴-10⁻⁶, 10⁻⁵-10⁻⁷, 10⁻³-10⁻⁶, 10⁻⁴-10⁻⁷, ranges therein, or combinations of such ranges).

In some embodiments, hydrogels of partially-denatured (e.g., electrostatically induced) albumin (e.g., HSA) have physical characteristics suitable for the variety of application described herein (e.g., device coating, drug delivery, tissue regeneration) as well as others. The hydrogels possess mechanical properties, such as bending strength, compression strength, tensile strength, shear strength, bending modulus, compression modulus, Young's modulus, etc. that make them suitable for various applications. In some embodiments, the mechanical properties of the hydrogels described herein are tunable based on, for example, conditions of formation (e.g., pH, ionic strength, concentration of protein, temperature, etc.), maintained conditions of the hydrogel (e.g., pH, ionic strength, concentration of protein, temperature, etc.), sequence of the albumin (e.g., HSA, BSA, mutant albumin, fragments thereof, etc.), formation of a composite/hybrid with another material, etc. In some embodiments, the hydrogels and composites thereof are such that they exhibit a preferred bending strength. In some embodiments, the composite has a bending strength of from about 10 to about 60 MPa (e.g., 10 MPa . . . 20 MPa . . . 30 MPa . . . 40 MPa . . . 50 MPa . . . 60 MPa, and ranges therein). In other embodiments, the hydrogels and composites thereof are such that they exhibit a preferred compression strength, wherein the compression strength is from about 20 to about 80 MPa (e.g., 20 MPa . . . 30 MPa . . . 40 MPa . . . 50 MPa . . . 60 MPa . . . 70 MPa . . . 80 MPa, and ranges therein). In some embodiments, the hydrogels and composites thereof are such that they exhibit a preferred tensile strength. In some embodiments, tensile strengths range of from about 2 to about 20 MPa (e.g., 2 MPa . . . 6 MPa . . . 10 MPa . . . 14 MPa . . . 18 MPa . . . 20 MPa, and ranges therein). In some embodiments, the hydrogels are characterized according to their shear strength. In some embodiments, the shear strength of the hydrogel is between about 15 to about 35 MPa (e.g., 15 MPa . . . 20 MPa . . . 25 MPa . . . 30 MPa . . . 35 MPa, and ranges therein). In some embodiments, the hydrogels and composites thereof are such that they exhibit a preferred bending modulus. The bending modulus may be from about 0.2 to about 0.6 GPa (e.g., 0.2 GPa . . . 0.3 GPa . . . 0.4 GPa . . . 0.5 GPa . . . 0.6 GPa, and ranges therein). In other embodiments, the hydrogels are characterized by having a compression modulus of from about 0.1 to about 0.5 GPa (e.g., 0.1 MPa . . . 0.2 MPa . . . 0.3 MPa . . . 0.4 MPa . . . 0.5 MPa, and ranges therein). In still other embodiments, the hydrogels may be characterized by a tensile modulus of from about 0.15 to about 0.4 GPa (e.g., 0.15 GPa . . . 0.2 GPa . . . 0.25 GPa . . . 0.3 GPa . . . 0.35 GPa . . . 0.4 GPa, and ranges therein). The hydrogels and composites thereof may be characterized by any one or more of these features.

In some embodiments, partially-denatured (e.g., electrostatically induced) protein (e.g., albumin (e.g., HSA, BSA, etc.)) hydrogels exhibit one or more of the characteristics of biodegradability, bioabsorbability, drug binding/releasing, biocompatibility, and/or non-toxicity.

### V. Preparation

Protein hydrogels are typically produced by extensive chemical crosslinking of the proteins or by thermal denaturation. In the case of thermal denaturation, the proteins must typically be extensively or totally denatured in order to produce a hydrogel. The addition of crosslinking agents or the total denaturation of the protein can have dramatic (e.g., negative) effects on the structure and importantly the function of the protein.

Provided herein are methods to partially denature proteins (e.g., albumin) electrostatically. For example, a protein (e.g., water soluble protein) at or near physiological pH (e.g., 6.5-8.5, 7.0-8.0, values and ranges therein, etc.) are subjected to a drop in pH (e.g., rapid, gradual, etc.) to below, for example pH 5.0 (e.g., 4.9, 4.8, 4.7, 4.6, 4.5, 4.4, 4.3, 4.2, 4.1, 4.0, 3.9, 3.8, 3.7, 3.6, 3.5, 3.4, 3.3, 3.2, 3.1, 3.0, 2.9, 2.8, 2.7, 2.6, 2.5, or lower). It is contemplated that the electrostatic disruption results in solvent exposure of otherwise solvent inaccessible hydrophobic residues within the protein. Then either at the lower pH or upon return to more neutral pH, hydrophobic attractions between the exposed hydrophobic residues of adjacent proteins result in protein aggregation and hydrogel formation. Despite the return to neutral or physiological pH, the local energy minima created by the hydrophobic interactions and protein aggregation prevent reversion to the native protein fold. It should be note that the present invention is not limited to any particular mechanism of action and an understanding of the mechanism of action is not necessary to practice the present invention.

In some embodiments, a starting solution of protein (e.g., albumin (e.g., HSA, BSA, etc.)) is provided in the neutral pH range (e.g., 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, and any values and ranges therein) at a concentration of between 1 mM and 100 mM (e.g., 1 mM . . . 2 mM . . . 3 mM . . . 4 mM . . . 5 mM . . . 6 mM . . . 7 mM . . . 8 mM . . . 9 mM . . . 10 mM . . . 15 mM . . . 20 mM . . . 30 mM . . . 50 mM . . . 75 mM . . . 100 mM, and any values and ranges therein).

In some embodiments, the pH of a neutral starting solution of protein (e.g., albumin (e.g., HSA, BSA, etc.)) is reduced by the addition of a suitable acid to produce an acidic hydrogelation solution. Examples include, but are not limited to: HCl, HNO₃, H₂SO₄, CHCO₂, HF_{(aq)}, etc. In some embodiments, the pH is reduced gradually, for example, by dropwise addition of acid. Gradual pH reduction may take place over 1 minute . . . 5 minutes . . . 10 minutes . . . 20 minutes . . . 30 minutes . . . 1 hour . . . 2 hours, or more. In other embodiments, the pH is reduced rapidly, for example, by rapid addition of a volume of acid (e.g., 1/100, 1/50, 1/25, 1/10, 1/4, 1/2, equal volume) to the neutral starting solution. Rapid pH reduction may take place in less than 1 minute . . . 30 seconds . . . 20 seconds . . . 10 seconds . . . 5 seconds . . . 2 seconds . . . 1 second, or less.

In some embodiments, proteins (e.g., albumen) are allowed to incubate in the acidic hydrogelation solution for a period of time (e.g., 1 minute . . . 2 minutes . . . 5 minutes . . . 10 minutes . . . 20 minutes . . . 30 minutes . . . 1 hour . . . 2 hours, or more) to allow complete hydrogel formation. In some embodiments, incubation occurs at, for example: 20° C. . . . 25° C. . . . 30° C. . . . 35° C. . . . 40° C. . . . 45° C. . . . 50° C. . . . 55° C. . . . 60° C. . . . 65° C. . . . 70° C. . . . 75° C. . . . 80° C.). In some embodiments, ionic conditions of the hydrogelation solution are adjusted to facilitate hydrogel formation. Various salts, including but not limited to NaCl, KCl, MgCl₂, etc. may be added to suitable concentrations (e.g., 10 mM . . . 20 mM . . . 50 mM . . . 100 mM . . . 200 mM . . . 500 mM . . . 1M, etc.). One of skill in the art will be capable of adjusting hydrogellation conditions accordingly.

In some embodiments, the pH of an acidic hydrogelation solution or that of a hydrogel produce therein is raised by the addition of a suitable base. Examples include, but are not limited to: NaOH, KOH, Ca(OH)₂, NH₃, CaO, etc. In some embodiments, the pH is raised gradually, for example, by dropwise addition of base. Gradual pH increase may take place over 1 minute . . . 5 minutes . . . 10 minutes . . . 20 minutes . . . 30 minutes . . . 1 hour . . . 2 hours, or more. In other embodiments, the pH is raised rapidly, for example, by rapid addition of a volume of base (e.g., 1/100, 1/50, 1/25, 1/10, 1/4, 1/2, equal volume) to the neutral starting solution. Rapid pH reduction may take place in less than 1 minute . . . 30 seconds . . . 20 seconds . . . 10 seconds . . . 5 seconds . . . 2 seconds . . . 1 second, or less.

In some embodiments, aggregation occurs in the low pH solution and hydrogel formation occurs. In such embodiments, upon return to neutral pH, the hydrogel structure remains, without return of the proteins to their native fold. In other embodiments, the electrostatic environment in the acidified solution stabilizes the partially-denatured protein (e.g., albumin) without significant aggregation. In such embodiments, upon return to neutral pH, the hydrogel structure forms, without return of the proteins to their native fold.

In some embodiments, the starting solution contain at least 14 wt % and less than 80 wt % protein (e.g., 14 wt %, 15 wt %, 16 wt %, 17 wt %, 18 wt %, 19 wt %, 20 wt %, 21 wt %, 22 wt %, 23 wt %, 24 wt %, 25 wt %, and values and ranges therein).

In some embodiments, the partially-denatured protein (e.g., albumen) hydrogel is allowed to incubate upon return to neutral solution for a period of time (e.g., 1 minute . . . 2 minutes . . . 5 minutes . . . 10 minutes . . . 20 minutes . . . 30 minutes . . . 1 hour . . . 2 hours, or more) to allow complete hydrogel formation. In some embodiments, post-hydrogellation incubation occurs at, for example: 20° C. . . . 25° C. . . . 30° C. . . . 35° C. . . . 40° C. . . . 45° C. . . . 50° C. . . . 55° C. . . . 60° C. . . . 65° C. . . . 70° C. . . . 75° C. . . . 80° C.). In some embodiments, ionic conditions of the post-hydrogelation solution are adjusted maintenance of the hydrogel. Various salts, including but not limited to NaCl, KCl, MgCl2, etc. may be added to suitable concentrations (e.g., 10 mM . . . 20 mM . . . 50 mM . . . 100 mM . . . 200 mM . . . 500 mM . . . 1M, etc.). One of skill in the art will be capable of adjusting post-hydrogellation conditions accordingly.

In some embodiments, a hydrogel is sterilized for various applications (e.g., medical, implantation, etc.). Any suitable method for sterilization may be utilized, including, but not limited to filtration, ethanol exposure, UV exposure, heat, etc.

In some embodiments, hydrogels are either formed in containers that impose a shape upon the hydrogel or are shaped post-formation. A hydrogel may be manipulated into any suitable shape (e.g., disk, tube, rod, sphere, particle, amorphous, irregular, oval, etc.) and size. Suitable dimensions include lengths, widths, heights, depths, and/or diameters of 1 mm . . . 2 mm . . . 5 mm . . . 1 cm . . . 2 cm . . . 5 cm . . . 10 cm, etc.

### VI. Particles

In some embodiments, provided herein are partially-denatured protein (e.g., albumin) hydrogel particles (e.g., micorparticles, nanoparticles). In some embodiments, these hydrogel particles range in size from 1 nm to 1 mm in diameter, length, width, depth, etc. In some embodiments, particles are microparticles or microspheres. In some embodiments, particles are nanoparticles or nanospheres. In some embodiments, particles are of any suitable shape (e.g., cylindrical, sicoidal, spherical, tabular, ellipsoidal, equant, irregular, etc.). In some embodiments, a population of partially-denatured protein (e.g., albumin) hydrogel particles is provided.

In some embodiments, a population of particles comprises individual partially-denatured protein (e.g., albumin) hydrogel particles of different shape (e.g., cylindrical, sicoidal, spherical, tabular, ellipsoidal, equant, irregular, etc.), size (e.g., nanoparticles and microparticles, different sized nanoparticles, different sized microparticles), and/or composition (e.g., salt concentration, albumin concentration, etc.). In some embodiments, a population of partially-denatured protein (e.g., albumin) hydrogel particles is a homogeneous population. In some embodiments, a population is 85% homogeneous, 90% homogeneous, 95% homogeneous, 99% homogeneous, of 99.9% homogeneous. In some embodiments, the size of a population of particles adopts a “normal distribution”, with very few particles having extreme dimensions and the majority having average diameters or very near average dimensions.

In some embodiments, hydrogel microparticles are provided with dimensions (e.g., length, width, height, depth, and/or diameter) of at least 1 μm and less than 1 mm. Suitable dimension include: about 1 μm, about 2 μm, about 5 μm, about 10 μm, about 20 μm, about 50 μm, about 100 μm, about 200 μm, about 500 μm, about 950 μm, and any dimensions or ranges therein. In some embodiments, hydrogel microspheres are provided with diameters of at least 11 μm and less than 1 mm. Suitable diameters include: about 1 μm, about 2 μm, about 5 μm, about 10 μm, about 20 μm, about 50 μm, about 100 μm, about 200 μm, about 500 μm, about 950 μm, and any dimensions or ranges therein.

In some embodiments, hydrogel nanoparticles are provided with dimensions (e.g., length, width, height, depth, and/or diameter) of at least 1 nm and less than 1 μm. Suitable dimension include: about 1 nm, about 2 nm, about 5 nm, about 10 nm, about 20 nm, about 50 nm, about 100 nm, about 200 nm, about 500 nm, about 950 nm, and any dimensions or ranges therein. In some embodiments, hydrogel nanospheres are provided with diameters of at least 1 nm and less than 1 μm. Suitable diameters include: about 1 nm, about 2 nm, about 5 nm, about 10 nm, about 20 nm, about 50 nm, about 100 nm, about 200 nm, about 500 nm, about 950 nm, and any dimensions or ranges therein.

The average diameter of particles may be determined by any suitable methods, such as fluidized bed separation and sieving, also called screen filtering. Particularly useful is sieving through a series of sieves appropriate for recovering samples containing microparticles of desired sizes. The average diameter can then be determined by techniques such as optical microscopy imaging using a graticule eye piece. In this way the average diameter of a particle can be determined, for instance by application of Equivalence Circle diameter. For particles which are substantially spherical, the average diameter relates to the actual diameter of the microsphere.

In some embodiments, hydrogel particels are produced by forming droplets of a desired size from a starting solution containing the protein to be partially denatured. Droplets may be formed by any suitable method. Aerosolization of the starting solution is method that is optionally utilized. Those droplets are then subjected to a hydrogelation procedure to form appropriately-sized hydrogel particles. In embodiments in which the hydrogel particles also contain one or more active agents or cells, the active agent or cell is either added to the protein prior to preparation of the starting solution, to the starting solution, to the droplets, or to the hydrogel particles.

In other embodiments, hydrogel particles are produced by subjecting a staring solution containing the protein to be partially denatured to a hydrogelation procedure. The bulk partially-denatured protein hydrogel is subsequently subjected to a particle forming procedure (e.g., aerosolization, mechanical production of appropriately-sized particles, drying, granulizing, etc.). In embodiments in which the hydrogel particles also contain one or more active agents or cells, the active agent or cell is either added to the protein prior to preparation of the starting solution, to the starting solution, to the hydrogel, or to the hydrogel particles.

In some embodiments, hydrogel particles are dried to obtain a powder or a film composition. For example, a spray drying or vacuum drying process can be carried out so that the hydrogel particles are dried into powder. Alternatively, the hydrogel particle composition may be sprayed or coated as a thin layer, and then the liquid of the composition is removed whereby forming a film of the hydrogel microparticle. Alternatively, the hydrogel particle composition can be mixed with solvent(s) so as to obtain a liquid, latex or gel containing the hydrogel particle composition.

In some embodiments, a device or system for producing hydrogel particles is provided. An exemplary device comprises one or more of: a starting solution reservoir, an aerosolization components, and acidification bath. Protein solution from the starting solution reservoir is aerosolized into the acidification bath wherein the hydrogelation occurs.

### VII. Pharmaceutical Preparations

In some embodiments, pharmaceutical preparations comprising partially-denatured albumin hydrogels are provided. In certain embodiments, partially-denatured albumin hydrogels find use in pharmaceutical preparations due to their ability to deliver low-solubility or toxic/harmful agents in a biocompatible manner. In certain embodiments, partially-denatured albumin hydrogels serve as carriers for difficult to deliver agents without using organic solvents. In such embodiments, the hydrogel pharmaceutical preparation comprises an active agent (e.g., therapeutic) for delivery (e.g., to an in vivo site) by elution from the hydrogel or degradation of the hydrogel in a physiologic environment. In some embodiments, an active agent is embedded or impregnated within the hydrogel (e.g., between partially-denatured albumins). In other embodiments, an active agent is contained within a reservoir (e.g., drug reservoir) within the hydrogel. Suitable therapeutics may include, but are not limited to: an antisense nucleotide, a thrombin inhibitor, an antithrombogenic agent, a tissue plasminogen activator, a thrombolytic agent, a fibrinolytic agent, a vasospasm inhibitor, a calcium channel blocker, a nitrate, a nitric oxide promoter, a vasodilator, an antimicrobial agent, an antibiotic, an antiplatelet agent, an antimitotic, a microtubule inhibitor, an actin inhibitor, a remodeling inhibitor, an agent for molecular genetic intervention, a cell cycle inhibitor, an inhibitor of the surface glycoprotein receptor, an antimetabolite, an antiproliferative agent, an anti-cancer chemotherapeutic agent, an anti-inflammatory steroid, an immunosuppressive agent, an antibiotic, a radiotherapeutic agent, iodine-containing compounds, barium-containing compounds, a heavy metal functioning as a radiopaque agent, a peptide, a protein, an enzyme, an extracellular matrix component, a cellular component, a biologic agent, an angiotensin converting enzyme (ACE) inhibitor, ascorbic acid, a free radical scavenger, an iron chelator, an antioxidant, a radiolabel, etc.

In some embodiments, in addition to administering the active agent and the hydrogel, the hydrogel pharmaceutical preparations containing suitable pharmaceutically acceptable carriers comprising excipients and auxiliaries which facilitate processing into preparations which can be used pharmaceutically.

Pharmaceutical preparations may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations provided herein are manufactured in a manner which is itself known, for example, by means of conventional mixing, granulating, dragee-making, dissolving, or lyophilizing processes. Thus, pharmaceutical preparations for oral use can be obtained by combining the active agents and partially-denatured protein hydrogels with solid excipients, optionally grinding the resulting mixture and processing the mixture of granules, after adding suitable auxiliaries, if desired or necessary, to obtain tablets or dragee cores.

Suitable excipients for use with hydrogel pharmaceutical preparations may include fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin. In addition, stabilizers may be added.

Possible pharmaceutical preparations which can be used rectally include, for example, suppositories, which consist of a combination of the partially-denatured protein hydrogel and one or more of the active agents with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include, for example, water-soluble salts and alkaline solutions. In addition, suspensions of the active compounds as appropriate oily injection suspensions may be administered. Preparations may contain substances which increase the viscosity and include, for example, sodium carboxymethyl cellulose, sorbitol, and/or dextran. Optionally, the suspension may also contain stabilizers.

Hydrogel pharmaceutical preparations may also find use in topical administration, and may be provided with carriers suitable for as much. Additionally, transdermal penetration enhancers can be employed in these topical formulations. Examples of such enhancers can be found in U.S. Pat. Nos. 3,989,816 and 4,444,762.

### VIII. Coatings

In some embodiments, partially-denatured albumin hydrogels are provided as a coating or surface of a device. For example, provided herein are methods and compositions for making and using implantable devices coated with partially-denatured albumin hydrogels or partially-denatured albumin hydrogels impregnated with therapeutic compositions and/or cells. Although coated implantable devices are discussed in greater detail below, embodiments are not limited to such devices, and the characteristics provided should be understood to apply to non-implantable devices as well.

The base material of the implantable device is prepared may be any base material that is typically used in medical devices, implants, prosthetic materials. Exemplary implantable devices may be prepared from one or more materials selected from the group consisting of: stainless steel, tantalum, titanium, nitinol, gold, platinum, inconel, iridium, silver, tungsten, a biocompatible metal, carbon, carbon fiber, cellulose acetate, cellulose nitrate, silicone, polyethylene terephthalate, polyurethane, polyamide, polyester, polyorthoester, polyanhydride, polyether sulfone, polycarbonate, polypropylene, high molecular weight polyethylene, polytetrafluoroethylene, a biocompatible polymeric material, polylactic acid, polyglycolic acid, a polyanhydride, polycaprolactone, polyhydroxybutyrate valerate, a biodegradable polymer, a protein, an extracellular matrix component, collagen, fibrin, a biologic agent, PEBAX, polyethylene, irradiated polyethylene or a suitable mixture, copolymer, or alloy of any of these.

Like the pharmaceutical preparations discussed above, the hydrogel coating may also comprise an active agent (e.g., therapeutic) for delivery (e.g., to an in vivo site) by elution from the hydrogel or degradation of the hydrogel in a physiologic environment. In some embodiments, an active agent is embedded or impregnated within the hydrogel (e.g., between partially-denatured albumins). In other embodiments, an active agent is contained within a reservoir (e.g., drug reservoir) within the hydrogel. Suitable therapeutics may include, but are not limited to: an antisense nucleotide, a thrombin inhibitor, an antithrombogenic agent, a tissue plasminogen activator, a thrombolytic agent, a fibrinolytic agent, a vasospasm inhibitor, a calcium channel blocker, a nitrate, a nitric oxide promoter, a vasodilator, an antimicrobial agent, an antibiotic, an antiplatelet agent, an antimitotic, a microtubule inhibitor, an actin inhibitor, a remodeling inhibitor, an agent for molecular genetic intervention, a cell cycle inhibitor, an inhibitor of the surface glycoprotein receptor, an antimetabolite, an antiproliferative agent, an anti-cancer chemotherapeutic agent, an anti-inflammatory steroid, an immunosuppressive agent, an antibiotic, a radiotherapeutic agent, iodine-containing compounds, barium-containing compounds, a heavy metal functioning as a radiopaque agent, a peptide, a protein, an enzyme, an extracellular matrix component, a cellular component, a biologic agent, an angiotensin converting enzyme (ACE) inhibitor, ascorbic acid, a free radical scavenger, an iron chelator, an antioxidant, a radiolabel, etc.

In some embodiments, the hydrogel coating also comprises cells contained within the hydrogel for delivery (e.g., to an in vivo site) by elution from the hydrogel or degradation of the hydrogel in a physiologic environment, or for growth on or within the hydrogel. In some embodiments, the coating acts as an extracellular matrix to support growth of cells. In some embodiments, it is impregnated with specific factors that facilitate growth of cells, e. g., growth factors, cytokines, chemokines and the like. In some embodiments, the coating comprises cells selected from the group consisting of endothelial cells, ligament tissue, muscle cells, bone cells, cartilage cells, smooth muscle cells, etc. In some embodiments, the coating supports the growth of the cells in vivo such that ultimately those cells are able to form part of the tissue site at which the device is implanted.

### IX. Biomedical Material

In some embodiments, provided herein are biomedical materials comprising or consisting of a partially-denatured albumin hydrogel (e.g., alone or with cells or one or more active agents therein), such as a wound dressing, a haemostatic material, or a drug delivery system.

In some embodiments, rather than providing a coating for a device made of another material, a device is made of a material comprising or consisting of a partially-denatured albumin hydrogel alone or with cells or one or more active agents therein. A hydrogel implant may be of any suitable shape (e.g., rod, disc, sphere, cube, flat square, irregular, shaped to fit a particular location in a body, etc.) and dimensions (e.g., length, width, height, diameter, etc. of: 1 mm . . . 2 mm . . . 5 mm . . . 1 cm . . . 2 cm . . . 5 cm . . . 10 cm, or more). In some embodiments, hydrogel implant finds use with any of the active agents or cells discussed above for coatings. In some embodiments, a hydrogel implant (or a coating) is provided as a hybrid or composite with another material (discussed below).

In some embodiments, a biomedical material comprising or consisting of a partially-denatured albumin hydrogel (e.g., alone or with cells or one or more active agents therein) is applied topically (e.g., on a wound, membrane, skin, tissue, etc.). In some embodiments, biomedical material facilitates delivery of active agents, wound healing, tissue regeneration, etc.

### X. Hybrids/Composites

In certain embodiments, hybrids or composites of the hydrogels described herein with other materials (e.g., other hydrogels, polymers, elastomers, bioceramics, etc.). Embodiments are not limited by the other types of materials that can be utilized with the hydrogels described herein.

In some embodiments, a hybrid or composite comprises a partially-denatured albumin hydrogel and a polymer selected from the group including, but not limited to: poly(diolcitrate), poly(hydroxyvalerate), poly(lactide-co-glycolide), poly(hydroxybutyrate), poly(hydroxybutyrate-co-valerate), polyorthoester, polyanhydride, poly(glycolic acid), poly(glycolide), poly(L-lactic acid), poly(L-lactide), poly(D,L-lactic acid), poly(D,L-lactide), poly(caprolactone), poly(trimethylene carbonate), and polyester amide.

In some embodiments, a hybrid or composite comprises a partially-denatured albumin hydrogel and a bioceramic selected from the group including, but not limited to: alcium phosphate bioceramics, alumina-based bioceramics; zirconia-based bioceramics; silica-based bioceramics, pyrolytic carbon-based bioceramics, and combinations thereof.

In some embodiments, hybrid materials and/or composites comprise between 1 and 99 wt % partially-denatured albumin hydrogel and correspondingly between 1 and 99 wt % of one or more other materials (adding to a total of 100 wt %). In some embodiments, materials comprise partially-denatured albumin hydrogel in an amount of 1 wt % . . . 5 wt % . . . 10 wt % . . . 15 wt % . . . 20 wt % . . . 25 wt % . . . 30 wt % . . . 35 wt % . . . 40 wt % . . . 45 wt % . . . 50 wt % . . . 55 wt % . . . 60 wt % . . . 65 wt % . . . 70 wt % . . . 75 wt % . . . 80 wt % . . . 85 wt % . . . 90 wt % . . . 95 wt % . . . 99 wt %, and ranges therein.

### EXPERIMENTAL

### Example 1

### Molecular Dynamics of Electrostatic Unfolding and Interactions of Albumin

Atomistic BSA Model Simulations.

A series of molecular dynamics (MD) simulations of bovine serum albumin (BSA) were conducted during development of embodiments of the present invention to develop a model of the protein at pH 3.5 and then used the model to investigate intermolecular interactions between two proteins. Fully atomistic MD simulations were performed using the GROMACS 4.5.4 simulation package (Berendsen et al. Comput. Phys. Commun. 1995, 91 (1-3), 43-56; Hess et al. J. Chem. Theory Comput. 2008, 4 (3), 435-447; Lindahl et al. J. Mol. Model. 2001, 7 (8), 306-317; Van der Spoel et al. J. Comput. Chem. 2005, 26 (16), 1701-1718; herein incorporated by reference in their entireties). The tertiary structure of BSA was first obtained by submitting the BSA primary sequence (GenBank: CAA76847.1) to a protein homology modeling server (CPHmodels 3.0) (Nielsen et al. Nucleic Acids Res. 2010, 38; herein incorporated by reference in its entirety). CPHmodels identified HSA as the closest existing protein structure to BSA and the result matches well (RMSD=1.39 Å) with recent crystallographic BSA structures (Bujacz, A. Acta Crystallogr., Sect. D: Biol. Crystallogr. 2012, 68, 1278-1289; herein incorporated by reference in its entirety). The resulting output file was used as the basis for all subsequent atomistic simulations of BSA. The all-atom optimized potential for liquid simulations (OPLS/AA) force field parameters (Jorgensen et al. J. Am. Chem. Soc. 1996, 118 (45), 11225-11236; herein incorporated by reference in its entirety) were used to describe interactions among the atoms. FAMBE-pH, a program that calculates the total solvation free energies of proteins as a function of pH, was used to calculate the ionization state of titratable residues (ASP, GLU, HIS, LYS, ARG) on BSA at pH 7.4 and 3.5.48 The 1:1 salt effect is included, indirectly, in the FAMBE-pH method as was done for the salt-dependent generalized Born method (Vorobjev et al. J. Phys. Chem. B 2008, 112 (35), 11122-11136; herein incorporated by reference in its entirety). Protonation states were fixed to the model for each pH and the model was then energetically stabilized by steepest descent algorithm, followed by an equilibration for at least 1 ns in water at 300K. The protein was then immersed in a 17×7×7 nm3 box of SPC water molecules (Berendsen et al. Ann. N.Y. Acad. Sci. 1986, 482, 269-86; herein incorporated by reference in its entirety) to allow room for protein expansion along the long axis of the simulation box, and a simulation was run for production for 64 ns in canonical (NVT) ensemble at constant temperature 300 K with Nose-Hoover temperature coupling method (Berendsen et al. Comput. Phys. Commun. 1995, 91 (1-3), 43-56; Jorgensen et al. J. Am. Chem. Soc. 1996, 118 (45), 11225-11236; herein incorporated by reference in their entireties). Analysis of protein secondary structures was performed by the STRIDE webserver (Heinig & Frishman. Nucleic Acids Res. 2004, 32, W500-W502; herein incorporated by reference in its entirety).

Circular Dichroism.

Dilute solutions (0.005 wt %) of essentially fatty acid free bovine serum albumin (A6003, Sigma, St. Louis, Mo.) in deionized water were titrated to different pH levels near the N—F transition (3.5, 4, 4.5) with HCl. Solutions were loaded into triple rinsed quartz cuvettes and evaluated by Circular Dichroism spectrography (J-815, JASCO Inc., Easton, Md.) with a wavelength scan from 190 to 260 nm in triplicate. Internal heating elements in the J-815 were used to thermally denature dilute albumin solutions (0.005 wt %) at pH 7.4 to 60 and 80° C.

Electrostatic Potential Calculations.

A Python script was written to compute the electrostatic potential explicitly (including all water and counterion molecules) at each point along the Connolly surface of the protein with the following equation:

$${\sum }_{i} \frac{1}{4 π {ɛ}_{0}} \frac{{Q}_{i}}{{r}_{i}}$$

where the sum runs over all atoms i that are within 3 nm from the point of the Connolly surface, Qi is the charge of atom i, and rᵢ is the distance between the charge i and the Connolly surface. The Connolly surface was computed using the built in GROMACS g_sas command with settings identical to those used by the program APBS51 embedded in Chimera (Pettersen et al. J. Comput. Chem. 2004, 25 (13), 1605-1612; herein incorporated by reference in its entirety) that was used to generate the Poisson-Boltzmann potential surface. The radius of the solvent probe was 1.4 Å with 20 dots per sphere on the surface. A 3 nm radius cutoff was used in calculating the electrostatic potential contribution of every atom near each mesh point. Visualizations of molecular structures are performed with the VMD 1.9.1 software package (Humphrey et al. J. Mol. Graphics Modell. 1996, 14 (1), 33-38; herein incorporated by reference in its entirety).

Results.

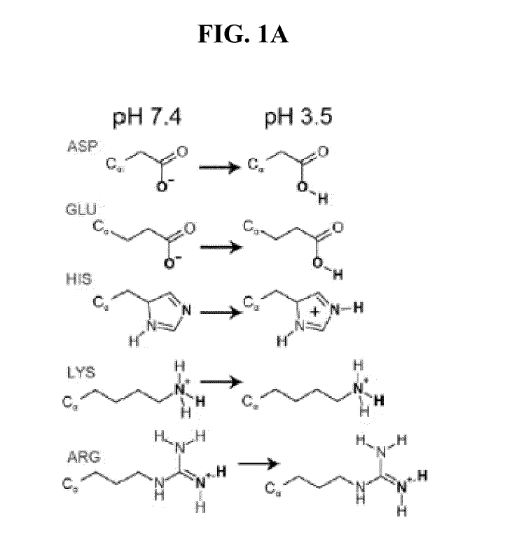

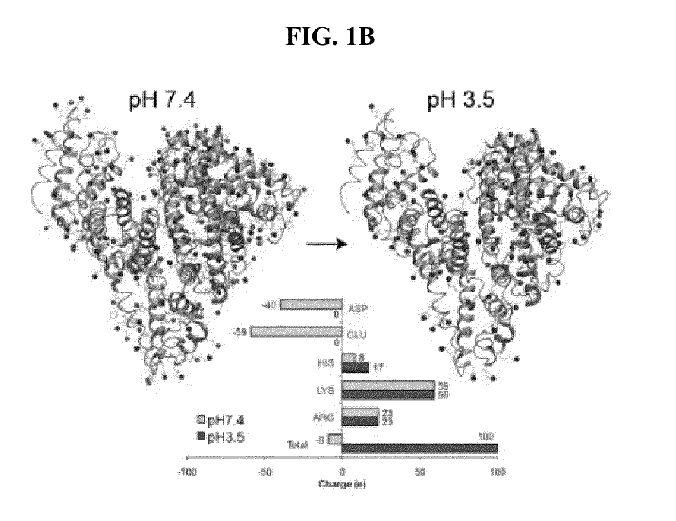

While the atomic structure of human albumin at pH 7.4 has been determined at a 2.5 Å resolution (Sugio et al. Protein Eng. 1999, 12 (6), 439-446; herein incorporated by reference in its entirety), only low-resolution 3D models based on X-ray scattering (SAXS) data exist for the F isoform (Leggio et al. Phys. Chem. Chem. Phys. 2008, 10 (45), 6741-6750; herein incorporated by reference in its entirety). Therefore, to study protein aggregation of F isoform bovine serum albumin at pH 3.5, an accurate model of the F isoform albumin was generated. Recent advances in computational power, MD software (Hess et al. J. Chem. Theory Comput. 2008, 4 (3), 435-447; herein incorporated by reference in its entirety), and theoretical methods to calculate titration states of residues in large proteins (Vorobjev et al. J. Phys. Chem. B 2008, 112 (35), 11122-11136; herein incorporated by reference in its entirety) now enable simulation of conformational changes from first principles. Since the size of the simulations required to model pH atomistically in this system remains prohibitively expensive, a program called FAMBEpH was used to calculate the total solvation free energies of proteins as a function of pH. This program employs a combination of approaches to calculate these free energies and involves (i) solving the Poisson equation with a fast adaptive multigrid boundary element method (FAMBE); (ii) calculating electrostatic free energies of ionizable residues at neutral and charged states; (iii) defining a precise dielectric surface interface; (iv) tessellating the dielectric surface with multisized boundary elements; and (v) including 1:1 salt effects. The computation of the free energy of solvation by FAMBE-pH includes the following terms: (1) the free energy of creation of a molecular cavity in the water; (2) the free energy of van der Waals interactions between the protein and the water solvent; (3) the free energy of polarization of the water solvent by the protein; and (4) the free energy of equilibrium titration of protein for a given pH and conformation.55 Since the number of ionizable groups in albumin (198) is more than ˜20-25, the Tanford-Schellman integral was used to calculate the equilibrium proton binding/release.48 With this program, we calculated the ionization state of titratable residues (ASP, GLU, HIS, LYS, ARG) at pH 3.5 (FIG. 1A). Residues with carboxylic acid groups that increase in charge state from −1 to 0 (ASP and GLU) between pH 7.4 and pH 3.5 are shown in red while residues with primary and secondary amines (LYS, ARG, and HIS) that increase in charge from 0 to +1 are shown in blue (FIG. 1). At pH 7.4, FAMBE-pH correctly predicted the deprotonation of all ASP and GLU residues: the protonation of all ARG and LYS residues, and a balance of protonated and deprotonated HIS residues were consistent with an expected overall net charge of −9 (FIG. 1A). At pH 3.5, FAMBE-pH predicted that all ASP and GLU residues become protonated and that the remaining HIS residues also be protonated, while LYS and ARG residues remain unchanged (FIG. 1B). The locations of these residues on BSA are distributed uniformly over the tertiary structure (FIG. 1B) and represent the ionization state of BSA at pH 3.5 (net charge of +100). While the pH of the protein was effectively set to 3.5, the conformational structure was still that of the N isoform. This predicted net charge was higher than the net charge (+65) and effective charge (+13) for albumin molecules at pH 3.5, as determined by experimental titration and electrophoresis NMR experiments (Bohme & Scheler. Chem. Phys. Lett. 2007, 435, 342-345; Tanford et al. J. Am. Chem. Soc. 1955, 77 (24), 6421-6428; herein incorporated by reference in their entireties), but this may be due to the fact that the structure of the protein was not yet in its ideal conformation. This difference can also be explained by the fact that any observable measurement should be computed from an ensemble of structures via a Boltzmann average, however, this is not feasible with the existent computational resources.

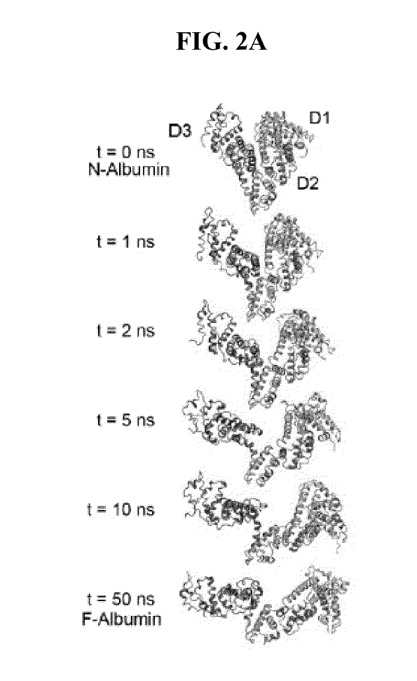

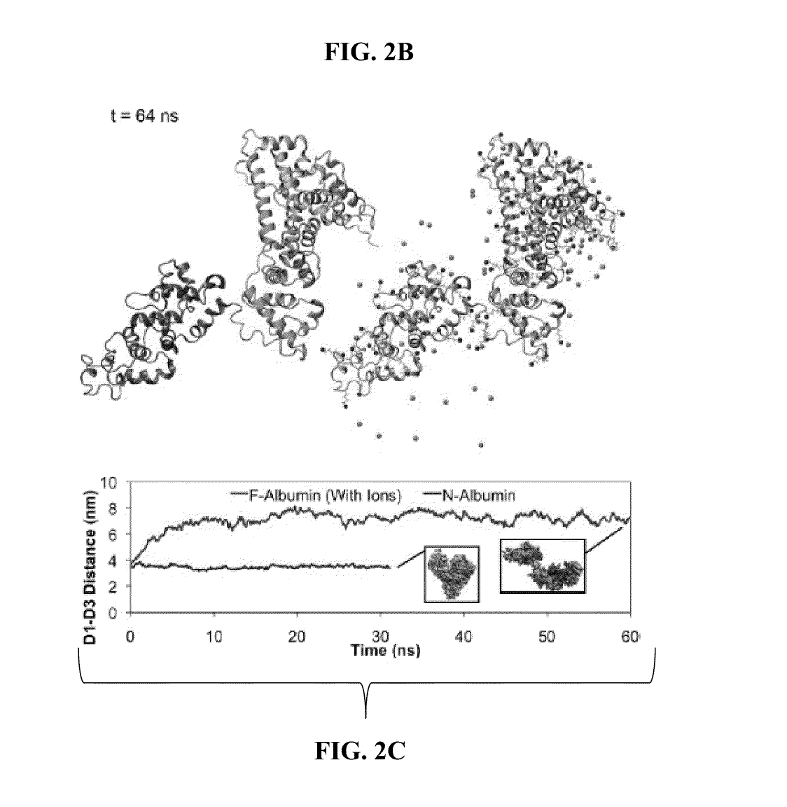

To produce the conformational changes induced by the change in the number of charges upon pH change, 100 neutralizing counterion charges and a large water box were added, and a large molecular dynamics simulation with ˜300 000 atoms was run. It was observed that the electrostatic repulsions between the three domains in the protein induced a conformational transition from the N isoform to an F-type isoform as shown in the simulation snapshots for the time evolution of this process in the presence of neutralizing counterions (FIG. 2A). Within tens of nanoseconds, the distance between domains 1 and 3 has increased, with the area between domain 2 and domain 3 acting as a hinge for the expansion as predicted in the literature (Geisow & Beaven. J. 1977, 163 (3), 477-484; Khan. J. 1986, 236 (1), 307-310; herein incorporated by reference in their entireties). After the initial expansion within this time, the conformation remained stable for up to 64 ns without significant conformational change (FIG. 2B). To quantify the simulated expansion, interdomain distances were measured between center of mass of domain 1 and domain 3 (FIG. 2C). Consistent with the simulation snapshots, the initial rate of protein expansion was ˜1.2 nm/ns. Final interdomain spacings of albumin was found to increase from 3.47±0.12 nm (N isoform) to 7.26±0.32 nm (F isoform) (FIG. 2C).

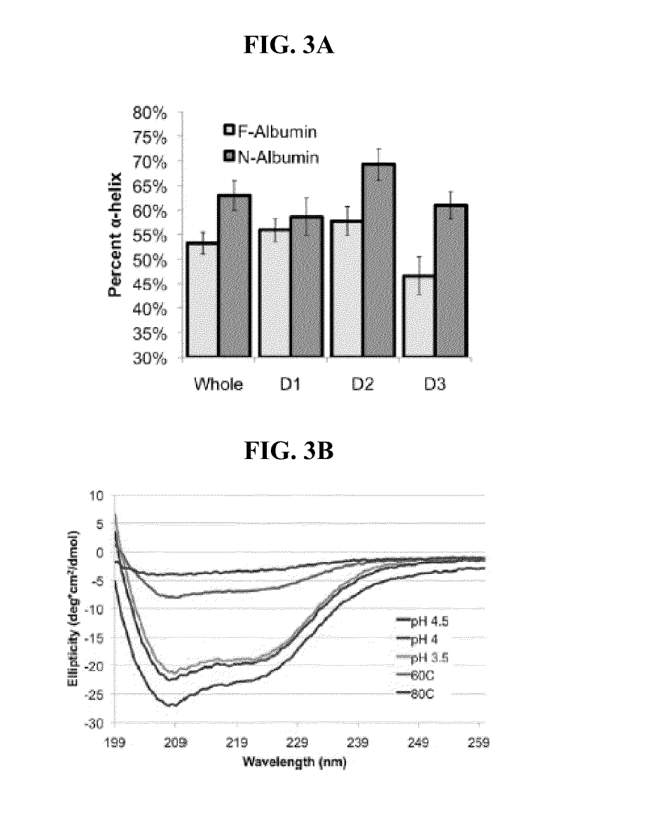

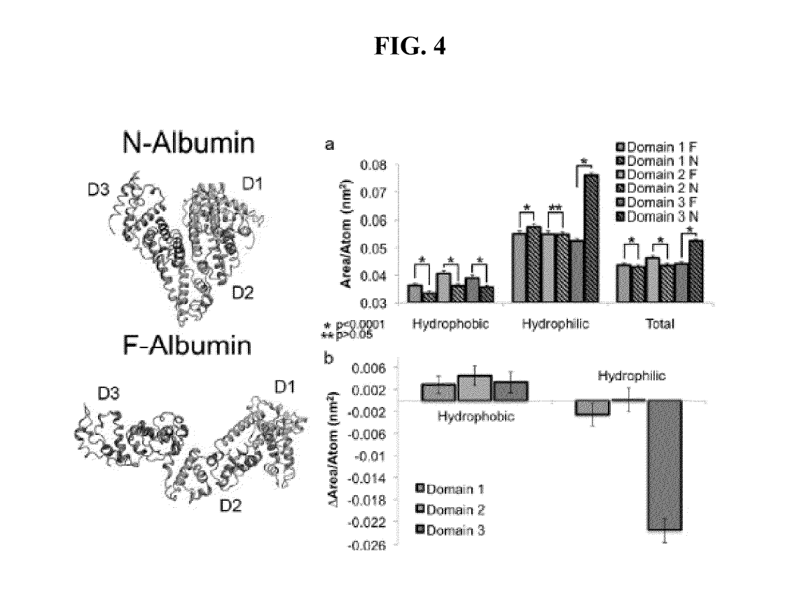

In addition to tertiary structural changes, the partial denaturation also resulted in a net loss of alpha helical secondary structure, from 62.9%±2.9% in the N isoform to 53.2%±2.2% in the F isoform (FIG. 3A). When resolved by domain, differences in the degree of preservation emerged. Domain 1 was the most preserved with a nonsignificant (p>0.05) decrease in alpha helical content from 58.6%±3.8% in the N isoform to 55.8%±2.4% in the F isoform. In contrast, both domains 2 and 3 had significant (p<0.01) decreases in helical content (domain 2: N=69.3%±3.8% to F=57.7%±3.7% and domain 3: N=61.0%±2.8% to F=46.6%±3.9%). Alpha helical signatures calculated from simulations were consistent with the presence of alpha helical signatures measured experimentally via circular dichroism spectroscopy at different pH values (3.5, 4, 4.5) in the F isoform range (FIG. 3B). In contrast, circular dichroism data for thermally denatured albumin near the limit (60° C.) and above (80° C.) albumin's denaturation temperature, reveals complete or near complete loss of all native secondary structures (FIG. 3B). Persistent secondary structural content in pH denatured albumin supports the notion that this partial denaturation pathway does not require disruption of the entire protein as in the case for thermally denatured albumin. The predicted and observed preservation of secondary structures further supports the use of hydrogels formed by the electrostatic triggering method of partial denaturation for drug delivery applications, particularly for drugs that utilize binding sites in domain 1. Having evaluated both tertiary and secondary structural changes during the N—F conformational transition, the effects of this transition at the individual residue level were then assessed. Specifically, the change in solvent exposure of hydrophobic residues was investigated to determine whether hydrophobic attractions might be present that could help explain the observed protein aggregation. Solvent accessible surface (SAS) area was calculated for each residue and categorized all residues as hydrophobic or hydrophilic as determined by the Serada et al. scale (FIG. 4) (Sereda et al. J. Chromatogr. A 1994, 676 (1), 139-153; herein incorporated by reference in its entirety). Measured SAS areas were normalized by the number of atoms contained within each category (domain 1/2/3 and hydrophobic/hydrophilic) for each of the (N and F) isoforms and report the absolute values (FIG. 4, panel a). The hydrophobic SAS for domains 1, 2, and 3 in the F isoform was 0.0362±0.0007 nm2/atom, 0.0406±0.0009 nm2/atom, and 0.0388±0.0011 nm2/atom, respectively. In the N isoform, the hydrophobic SAS for domains 1, 2, and 3 was 0.0333±0.0008 nm2/atom, 0.0361±0.0008 nm2/atom, and 0.0355±0.0007 nm2/atom, respectively. The analysis demonstrates that all three domains have a statistically significant (p<0.0001) increase in the SAS area of hydrophobic residues during the N—F transition. The differences between these absolute SAS area values (domain 1: 0.0028±0.0016 nm2/atom; domain 2:0.0045±0.0018 nm2/atom; domain 3: 0.0033±0.0019 nm2/atom) during the N—F transition reiterate the increase in hydrophobic SAS area for each domain (FIG. 4, panel b). In contrast, the SAS area of hydrophilic residues decreased significantly during the N—F transition. Hydrophilic SAS for domains 1, 2, and 3 in the F isoform was 0.0550±0.0010 nm2/atom, 0.0549±0.0012 nm2/atom, and 0.0524±0.0011 nm2/atom respectively. In the N isoform, hydrophilic SAS for domains 1, 2, and 3 was 0.0576±0.0010 nm2/atom, 0.0548±0.0010 nm2/atom, and 0.0760±0.0009 nm2/atom respectively. From a physical point of view of the entire protein, the hydrophobicity increases by 16% and the hydrophilicity decreases by 13%.

The total SAS area measurements when both hydrophobic and hydrophilic residues are taken together can be used to infer whether the individual domains are expanding or collapsing (FIG. 4, panel a). Although all of the N—F differences were different, the difference in domain 1 was modest (N=0.0429±0.0007 nm2/atom, F=0.0436±0.0007 nm2/atom). This small change is consistent with the earlier result that the change in alpha helical content was not significantly different between the two isoforms. However, the domain 2 expanded (N=0.0435±0.0007 nm2/atom, F=0.0462±0.0009 nm2/atom) and domain 3 collapsed (N=0.0524±0.0006 nm2/atom, F=0.0441±0.0009 nm2/atom) to a greater degree during the transition.

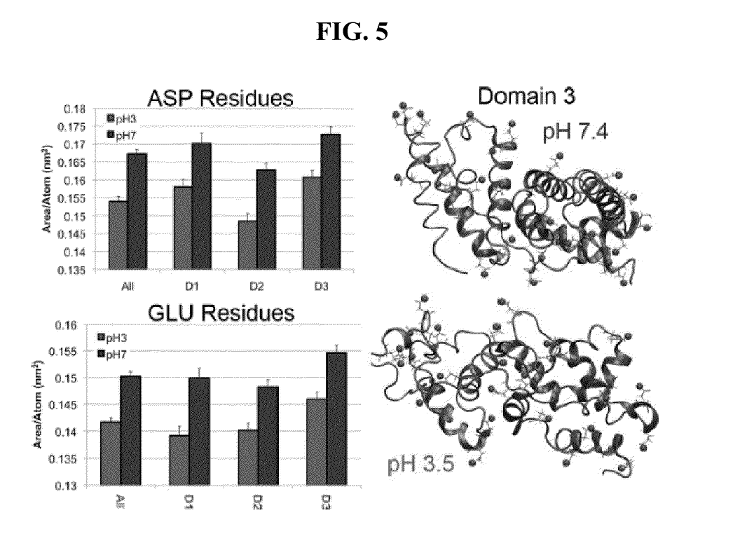

The large decrease in hydrophilic SAS area measured for domain 3 is worth noting. This effect is likely due to several reasons; first is the fact that ASP and GLU residues are protonated at pH 3.5 and thus, less hydrophilic, and second is the greater loss of secondary structure in domain 3. Taken together, these two effects allow ASP and GLU residues to become buried, reducing their SAS area contribution (FIG. 5). While ASP and GLU residue SAS areas decrease in every domain, they are disproportionately represented in domain 3, making these effects more noticeable. On the whole, the protein is more hydrophobic in the F isoform than in the N isoform. The increases in hydrophobic SAS area and decreases in hydrophilic SAS area suggest that aggregation of F isoform BSA molecules in high concentrations may be due to intermolecular hydrophobic interactions.

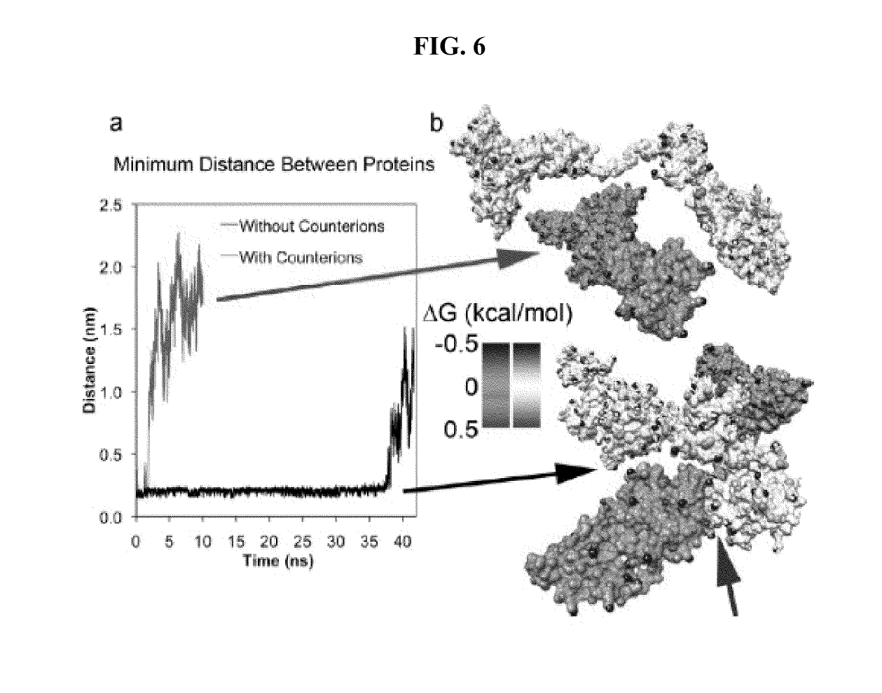

Experiments were conducted during development of embodiments of the present invention to investigate the interactions between two proteins using the new F isoform albumin models. Two of these configurations were placed in contact such that their newly exposed hydrophobic surfaces, as determined by the increase in local hydrophobic SAS, were facing each other. With this arrangement, the effective concentration of albumin in water in this simulation was ˜7 mg/mL, substantially lower than the experimentally observed threshold for gelation (15 mg/mL) but sufficient for examining the interaction between two proteins. Two types of simulations were run, one with explicit counterions and the other without them. The absence of counterions, while unphysical, results in a tremendous speed up of the simulations and the aim was to check whether physical insightful results could be obtained. However, in the absence of counterions, large electrostatic repulsions between the proteins forced them to move away from each other soon after overcoming the initial contact attraction (FIG. 6, panel a), leading to a result that is qualitatively wrong, as shown next, demonstrating the importance of appropriately counting for the explicit counterions.

In the presence of counterions necessary to maintain system electroneutrality (200 Cl—), the two proteins stayed within 0.25 nm of each other, as indicated by the minimum distance measured between the two proteins (FIG. 6, panel a). The persistent point of contact between the two proteins was located in domain 2 but this may be an artifact of the initial protein placement (FIG. 6, panel b). Interestingly, after 36 ns, the two proteins separated from each other. This suggests that the attraction observed between the two proteins may be a result of a local minimum in the free energy as a result of the increased hydrophobicity but would need to be corroborated with additional simulations.

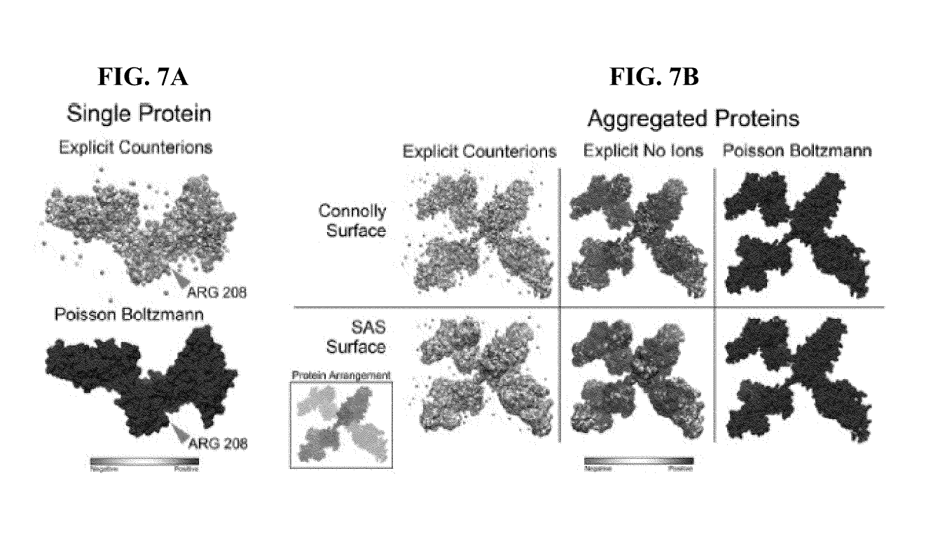

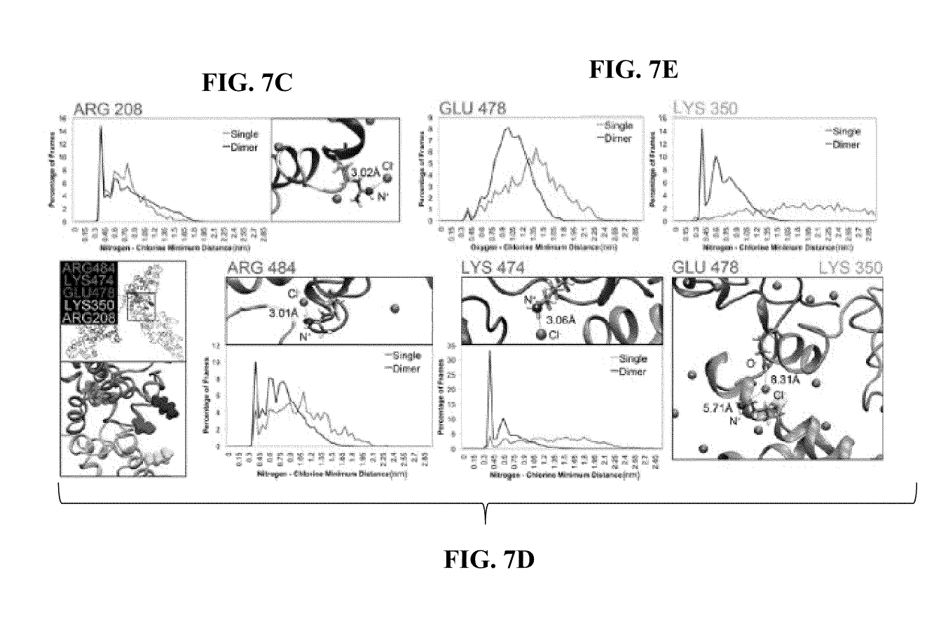

Calculation of the electrostatic surface energy potential provides an additional method to evaluate the intermolecular interactions. The usual way to determine electrostatic potentials in proteins is by solving the Poisson-Boltzmann (PB) equation. However, it is not clear how good the mean-field approximation would be in a system with such larger number of charges. Therefore, PB calculations were performed and explicit determination of the electrostatic potentials from the findings of the positions of all the molecules, including the ions, from the simulations. Explicit electrostatic potential calculations that factor the contribution of counterions in the system results in surface potentials that are more negative when compared to the result from PB (FIG. 7A). Particularly interesting is that, in the scale shown in FIG. 7a, the PB results show an almost constant, relatively high, positive potential that directly reflects the charge on the proteins, that is, the +100 that result from the low pH. In sharp contrast, the explicit calculations demonstrate relatively large, variation of the electrostatic potential across the protein surface, showing that the explicit positions of the counterions plays a dramatic role in determining the structure and interactions of proteins. This is very important since the PB calculations would suggest strong attractive interactions between the protein and negatively charged molecules, or surfaces. On the other hand, the full calculations show a much more complex surface that could lead to a variety of possible interactions.

While in many cases the PB calculation is sufficient, it misses many important details regarding the effect of individual counterions in highly charged systems. For example, at residue ARG 208 (FIG. 7A), PB predicts the nitrogen atom to have a positive electrostatic potential. In fact, the explicit calculation indicates the potential is negative due to the attraction of a neighboring Cl⁻ counterion (FIG. 7C, right). A histogram of the distances to the nearest Cl⁻ ion for the charged N≧atom on ARG 208 demonstrates that this residue is typically bound to a counterion (FIG. 7C, left).

In the case of two proteins interacting with each other, similar effects were observed for the electrostatic surface potential calculation as in the single protein case (FIG. 7B). To underscore the important contribution of these counterions on the interpretation of the electrostatic potential, the explicit electrostatic potential was computed while ignoring the counterions present (FIG. 7B center). This results in a relatively high, positive potential similar to the one calculated by PB (FIG. 7B right). The potentials calculated at the Connolly surface (0.14 nm) and the SAS surface (1.4 nm) also demonstrate how the potential becomes more negative further from the positive charges on the protein.

Four additional residues at the point of contact between the two proteins are highlighted for further analysis (FIG. 7d). All four (and two in particular, LYS 350 and LYS 474) rarely had any associated counterions in the single protein case. But, when brought in contact with another highly charged protein, all four residues were substantially more likely to have counterions present (FIG. 7e). Both LYS 350 and LYS 474 were rarely seen without a counterion present after dimerization. In the case of GLU 478 and LYS 350, a chlorine ion was found close to both proteins (orange and purple). While there are many other positively charged surface residues on both proteins, they do not all recruit counterions to them as in the case of LYS 350 and LYS 474. This is due to the inherent entropic cost of binding every free counterion with every positively charged residue but it becomes more likely when the proteins are dimerized (FIG. 7e). The increased likelihood of finding nearby counterions in the dimerized state suggests the attraction of these counterions is necessary to neutralize residue charges and promote protein aggregation.

The above observations indicate that hydrophobic interactions from the protein core and counterion association to charged residues at the proteins point of contact drives the self-assembly of the hydrogel network. Importantly, the electrostatically driven denaturation observed in these fully atomistic BSA simulations captures the conformational structures predicted by others in the literature (Carter & Ho. Adv. Protein Chem. 1994, 45, 153-203; Leggio et al. Phys. Chem. Chem. Phys. 2008, 10 (45), 6741-6750; herein incorporated by reference in their entireties) but with a much greater accuracy.

### Example 2

### Albumin Hydrogels Formed by Electrostatically Triggered Self-Assembly and Drug Delivery Capability Thereof

Materials and Hydrogel Synthesis.

BSA gel precursor solutions were formed by adding deionized water to essentially fatty acid free bovine serum albumin (A6003, Sigma, St. Louis, Mo.) in concentrations ranging from 9-20 wt % (1.4-3 mM). Solutions were stirred at 200-300 RPM until complete dissolution (˜2-3 hours). To form 93 pH-induced bovine serum albumin gels (PBSA), the pH of the precursor solution was lowered to pH 3.5 by drop wise addition of 2M HCl with constant stirring followed by submersion in a water bath at 37° C. for 2 hours. To form thermally denatured bovine serum albumin gels (TBSA), the precursor solution was neutralized to pH 7.4 by 2 M NaOH followed by submersion in a water bath at 80° C. for 2 hours. Precursor solutions were sterilized with a 0.2 μm nylon syringe filter before gelation (Fisher Scientific, PA).

Atomistic BSA Model Simulations.

Molecular dynamics simulations were performed on BSA whose protonation state was set to pH 3.5 to obtain the structure of partially denatured BSA, as determined by FAMBE-pH, a program that calculates the total solvation free energies of proteins as a function of pH (Vorobjev et al. J Phys Chem B. 2008; 112(35):11122-36; herein incorporated by reference in its entirety). After randomly adding 10 molecules of atRA to the system, molecular dynamics simulations were performed using the GROMACS simulation package at constant temperature (300K) and pressure (l atm) (Hess et al. J Chem Theory Comput. 2008; 4(3):435-47; Van der Spoel et al. J Comput Chem. 2005; 26(16):1701-18; Lindahl et al. J Mol Model. 2001; 7(8):306-17; Berendsen et al. Comput Phys Commun. 1995; 91(1-3):43-56; herein incorporated by reference in their entireties). The OPLS/AA force field was used to simulate the atomistic BSA model (9336 atoms) solvated with ˜94,000 SPC water molecules, 100 counterions, and 10 atRA molecules (Berendsen et al. Comput Phys Commun. 1995; 91(1-3):43-56; Jorgensen et al. J Am Chem Soc. 1996; 118(45):11225-36. herein incorporated by reference in their entireties). The atRA-BSA models were equilibrated by a steepest descent algorithm followed by a 100 ns NVT production run with periodic boundary conditions.

Potential of Mean Force Calculation.

The potential of mean force was calculated from a series of umbrella sampling (Torrie et al. Chemical Physics Letters. 1974; 28(4):578-81; herein incorporated by reference in its entirety) simulations where configurations of bound atRA molecules were placed at linearly increasing distances (Δz=0.5 Å) from their self-selected preferred binding pocket after 100 ns of unconstrained molecular dynamics. The center of mass of the atRA molecule and the center of mass of neighboring binding pocket residues were used as the anchor points to determine the separation distance z between the BSA binding site and atRA. The potential of mean force was then calculated on the output of the 30 simulations after 5 ns using the a weighted histogram analysis method (g_wham) embedded in the GROMACS software (Hub et al. J Chem Theory Comput. 2010; 6(12):3713-20; herein incorporated by reference in its entirety). The number of configurations and separation distances were selected such that the entire phase space was sufficiently sampled until z is at least 2 nm from the binding site where z=0 is the preferred binding distance after 100 ns.

Cryo-SEM Imaging.

PBSA and TBSA gels were vitrified with a controlled environment vitrification system (CEVS)[Bellare et al. J Electron Micr Tech. 1988; 10(1):87-111; Issman & Talmon. J Microsc-Oxford. 2012; 246(1):60-9; herein incorporated by reference in their entireties. The CEVS consists of four modules: (1) temperature control module, (2) environmental chamber, (3) plunge module and (4) cryogen box. CEVS is operated in a fume hood, to pump away nitrogen gas and possibly small amounts of ethane vapour while maintaining the room air-conditioned at 20 C to reduce ambient humidity. Samples (3 μL) are loaded onto planchettes and a copper grid is immersed in the sample. A second planchette is placed over the sample facedown making a spring-loaded planchette sandwich and is then locked into a plunging tweezer module. A cable release mechanism simultaneously opens a trap door in the CEVS and plunges the tweezers with the planchette sandwich into the cryogen box that contains liquid ethane in an LN2 bath. The planchette sandwich is then moved from the liquid ethane into the LN2 bath and transferred to a BAL-TEC BAF060 for further processing (BAL-TEC, Austria). The spring-loaded planchette sandwich is opened which fractures the sample surface and then freeze etched by raising the temperature to sublimate water from the surface. A thin Pt/C coating several nm thick is coated onto the fracture surface and then transferred to a Zeiss Ultra plus HR-SEM for Cryo-SEM imaging (Zeiss, Thornwood, N.Y.).

Mechanical Indentation of BSA Gels.

A custom built microindenter was used to measure the Young's modulus of BSA gels (Lin et al. J Mater Res. 2009; 24(3):957-65; herein incorporated by reference in its entirety). A flat-ended cylindrical stainless steel punch with a radius a=0.44 mm was used to indent the surface of the gel with a Burleigh inchworm motor (Rochester, N.Y.) attached to a Sensotec 1 kg load cell (Columbus, Ohio) while the displacement was measured with a Philtec optical displacement sensor (Annapolis, Md.). As the probe indented the sample at a fixed rate (10 μm/s), the load was recorded on a computer. The following equation is the relationship between the load P and displacement 6 in the linear regime of the curve:

$$\frac{P}{δ} = \frac{8 aE}{3 {f}_{c} ( a / h )}$$

This relationship can be used to convert the recorded loads into stresses for determination of the Young's modulus. The term, f_{c} is a geometric confinement factor determined by the ratio of the indenter radius to the gel thickness h which, in this work, is ˜1. Rewriting Eq. 1 yields an expression for the average stress σ_{avg} under the indenter:

$${σ}_{avg} = \frac{P}{π {a}^{2}} = \frac{8 E}{3 π} ( \frac{δ}{a} ) \frac{1}{{f}_{c} ( a / h )}$$

The slope of the curve in the linear regime can be used to calculate the Young's modulus E during the indentation. For low values of a/h, where f_{c}=1, the quantity functions as the effective strain. All differences (between conditions and concentrations) are significant at p<0.001 levels.

Rheological characterization of BSA gels. PBSA (pH 3.5) and TBSA precursor solutions were made at several concentrations (20, 18, 16, 14, 12 wt %). The amount of water added to each polymer solution was calculated to give the final polymer concentration. Rheometric characterization was performed on Discovery Hybrid Rheometer (TA Instruments, New Castle, Del.) equipped with a Peltier hood and evaporation blocker. Samples were heated to either 37° C. (PBSA) or 80° C. (TBSA) to evaluate the gelation kinetics for both gel types. Small 0.5% oscillatory strain at was applied throughout the experiment while measuring the sample storage and loss modulus over time. The onset of hydrogel formation is defined as the crossover between the storage and loss modulus.

In Vivo Subcutaneous Rat Model.

Eight female Sprague-Dawley rats (Harlan Laboratories, Inc.) weighing 150-175 g were used for in vivo biocompatibility testing of the BSA gels. Four rats were randomly assigned into two groups for explant time points at 4 days and 4 weeks for evaluation of the acute and chronic inflammatory response. Animals were anesthetized using the inhalant machine Impact 6 (Vetequip Inc., Pleasanton, Calif.). Isofluorane was administered at a concentration of 2% with an oxygen flow rate of 2 L/min. Following anesthesia; the backs of the animals were shaved and then disinfected with butadiene followed by alcohol and a second butadiene wipe. Two incisions of approximately 1.5 cm in length were made at the implantation sites and subcutaneous pockets were created by blunt dissection in each location. 20 wt % Albumin hydrogels 184 (both PBSA and TBSA) were fabricated as described in the methods section 2.1 and were cut into disks (h=0.5 cm, r=0.6 cm) using a sterile biopsy punch. In one location, an acid leached 20 wt % PBSA gel disk was implanted into the subcutaneous pocket far from the incision site. At the other incision site, a 20 wt % TBSA gel disk was implanted. A control saline injection and pH 7.4 (20 wt %) BSA solution was injected into the back of the rat in the two remaining implantation sites. Each disk or injection had a volume of 0.5 mL. In all, each rat received all four treatments (PBSA, TBSA, BSA solution, saline) in four different rotating locations (anterior right, anterior left, posterior right, posterior left) for both time points (4 day and 4 week). The wounds were closed with surgical staples and implants were subsequently removed after 4 days. At each time point, four animals were anesthetized and subsequently euthanized via CO2 asphyxiation. Cervical dislocation was performed as a secondary euthanasia method and the explants were harvested. The explants, which included the tissues surrounding the implanted material, were snap frozen in a dry ice/acetone mixture. Explants were stored at −80° C. until sectioning and H&E staining Stained sections were photographed in series and in adjacent regions along the dorsoventral axis from the interior of the implant to the skin surface. The Northwestern University Animal Care and Use Committee approved all animal procedures used in this work.

atRA Binding and Release.

All trans retinoic acid (atRA, 82625, Sigma, St. Louis, 206 MO) was added to solutions of BSA with molar ratio concentrations including 0:1, 0.08:1, 0.1:1, 0.13:1, 0.2:1, 0.4:1, 0.8:1, 1.2:1, 1.6:1, and 2:1 (atRA:BSA). A baseline fluorescence intensity of BSA at pH 7.4 and 3.5 measured at 340 nm with excitation at 295 nm was recorded (n=4 for each molar ratio concentration) as reported in the literature (Maiti et al. Int J Biol Macromol. 2006; 38(3-5):197-202; herein incorporated by reference in its entirety) using the plate reader Tecan Safire II (Tecan, Maennedorf, Switzerland). Then, atRA binding to BSA at pH 7.4 and 3.5 was assessed immediately after the addition of atRA at the different molar ratios. For measuring atRA release from hydrogel disks, a higher molar ratio concentration of 8:1 (atRA:BSA) was used when incorporating atRA into BSA precursor solutions before gelation. Precursor solutions were processed as normal to fabricate atRA-loaded PBSA and TBSA hydrogel disks with a final volume of 0.5 mL. Disks were submerged in 10 mL PBS at 37° C. for atRA release studies. Eluates were collected and replaced at 1, 3, 6, 12, 24, 48, 72, 144, and 240 hour sampling times and fluorescence intensity of atRA at 340 nm and BSA at 280 nm was collected with a NanoDrop™ 2000 C Spectrophotometer (ThermoScientific™, Waltham, Mass.).

Scratch Test Migration Assay.

Human aortic smooth muscle cells (HASMC) (Lonza, Basel, Switzerland) (passage 5) were cultured in SmGM-2 media. All cells were cultured at 37° C. in a humidified incubator containing 5% CO2. HASMCs (seeding density 1×104 cells/cm2) were seeded onto TCP surfaces and grown until 90% confluent. Cell culture media was changed every 2 days until confluence after which serum-free SmGM-2 media was used to create a nutrient starved environment for 24 hrs. A vertical scratch was made with a sterile 200 uL pipette tip in the confluent HASMC layer, rinsed with warm PBS, and replaced with 1 mL SmGM-2 media. The underside of the dish was marked near the wound area to aid in identification. Wells were randomly placed into 4 groups which received either a 100 uL dose of eluted material from atRA loaded PBSA or TBSA release study at day 10, a 100 uL dose of 24 ng atRA dissolved in PBS, or a control 100 uL of PBS. A light microscope (Nikon Eclipse TE2000-U) was used to capture images using Image Pro 5.0 software (MediaCybernetics, Bethesda, Md.) of the wound area immediately at day 0 and at various times until the control wound closed at 24 hrs. Wound areas were determined using an automated wound area measurement macro with the ImageJ 1.43r software (NIH, Bethesda, Md.). The measurement of migration was determined by subtracting the cell-free area at day 0 from the cell-free area at 24 hrs. Data were presented as means±SD of several independent experiments from each atRA-loaded hydrogel replicate.

### Example 3

### Fabrication of Albumin Hydrogels by Electrostatically Triggered Self-Assembly

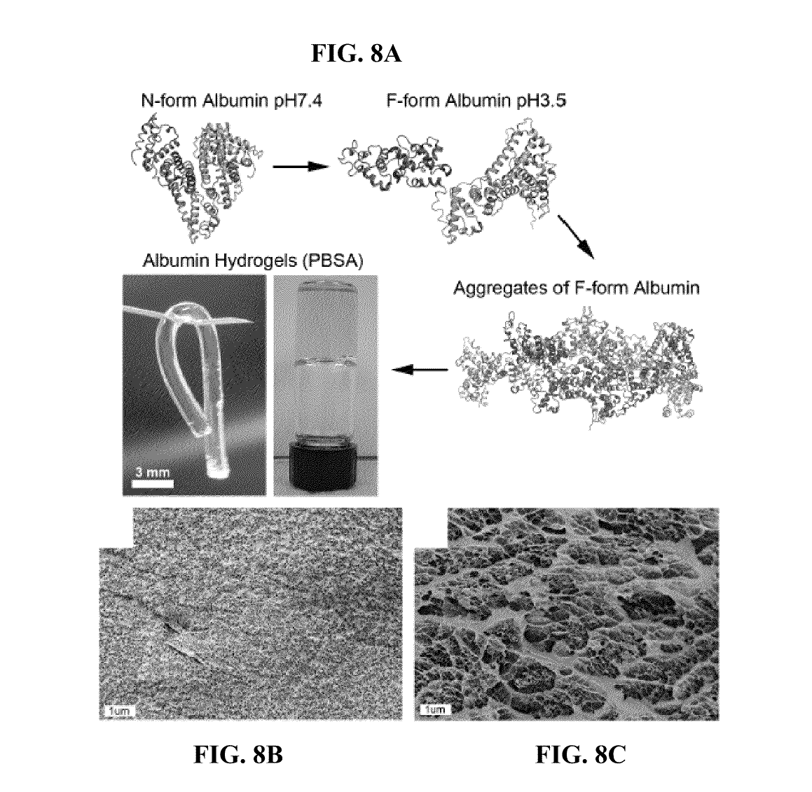

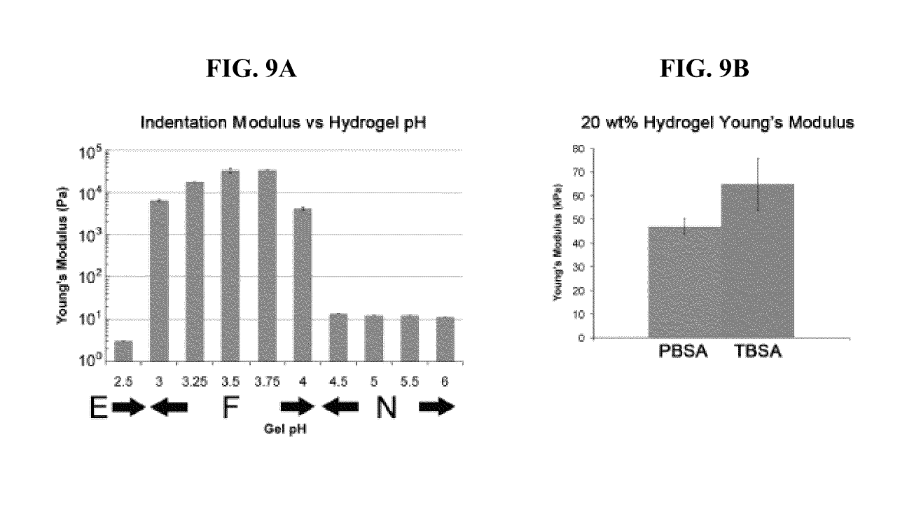

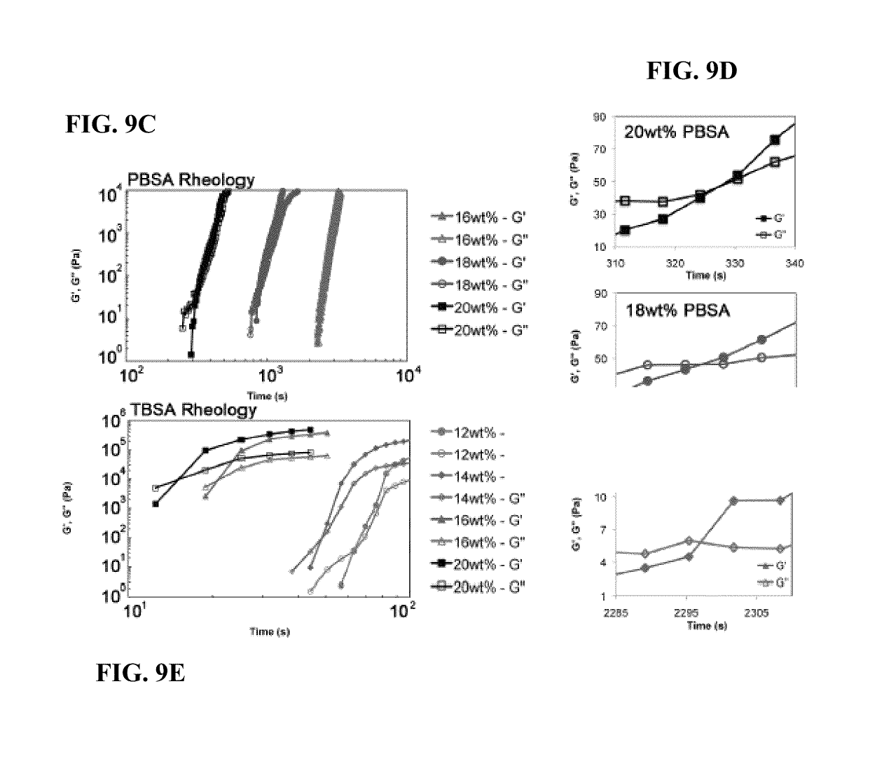

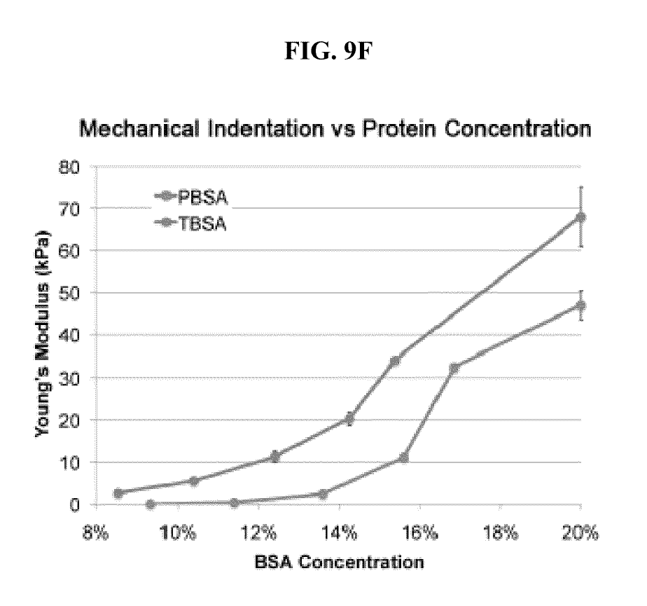

Albumin dissolved in de-ionized water formed a clear yellow solution. During drop wise addition of 2 M HCl, transient changes in turbidity were observed visually as the N-form albumin partially denatured to the F-form albumin, represented by the model of albumin undergoing this transition (FIG. 8A). In concentrated solutions >15 wt % with an optimal final pH 3.5 (FIG. 9), these partially denatured structures aggregate together and self-assemble into a solid hydrogel network within ˜24 hours at room temperature or in 10 minutes at 37° C. (FIG. 8A). Accelerated 10 minute hydrogel formation was achieved by placing small volumes of partially denatured albumin solutions (˜100 μL) inside tubular molds (3 mm diameter) in the 37° C. water bath with the effect of accelerated thermal equilibrium. Fabrication of larger volumes of albumin solutions >500 μL were made in cell culture wells or scintillation vials and submerged in 37° C. for 2 hours to ensure hydrogel formation. In contrast, BSA precursor solutions at pH 7.4 do not exhibit any gelation behavior unless the temperature rises above 62° C. to achieve thermal denaturation of the N-form. While PBSA and TBSA hydrogel appear identical at the macroscale, Cryo-SEM imaging reveals stark differences between the two hydrogels. FIG. 8B shows PBSA hydrogels to have a compact structure with small pores while FIG. 8C shows TBSA hydrogels to have an expanded structure with larger pore sizes.

To characterize the ideal pH range where PBSA hydrogels can form, hydrogels were subjected to compressive mechanical indentation testing to measure their Young's moduli. The strongest PBSA hydrogels were formed in the F-form pH range between 3.0 and 4.0 (FIG. 9A). Above pH 4, the transition from the N-form albumin to the F-form albumin was incomplete and these solutions did not form solid PBSA gels. Below pH 3.0, BSA gel solutions became highly viscous but never formed a solid gel. Maximal gel modulus (34 kPa) was achieved at pH 3.5 for 17 wt % PBSA hydrogels. Comparison of the Young's modulus between PBSA and TBSA hydrogels with the same protein concentrations indicates that the TBSA hydrogels are stronger than the PBSA hydrogels (FIG. 8B). 20 wt % PBSA and TBSA hydrogels were used for the remainder of this work with Young's modulus values of 46 kPa and 67 kPa, respectively. Rheological characterization of PBSA and TBSA samples in different concentrations demonstrate the gelling kinetics for the two types (FIG. 8 C, D, E). PBSA hydrogels in several different concentrations (16, 18, 20 wt %) form after several minutes (2301.7 s, 887.8 s, 330.5 s) as defined by the crossover between the G′ and G″ curves (FIG. 8 C, D) after temperature reaches 37° C. The PBSA hydrogels at the 12 wt % and 14 wt % concentration never had a crossover after 2 hours of testing (not shown). This finding delineates a critical minimum protein concentration of ˜15 wt % albumin for PBSA hydrogel formation. In contrast, the TBSA hydrogels in several different concentrations (12, 14, 16, 18, 20 wt %) formed very quickly (69.5 s, 50.7 s, 25.2 s, 18.8 s, 17.9 s) as defined by the crossover between the G′ and G″ curves (FIG. 8 E) after temperature reaches 80° C. For clarity, the crossover curves representing 18 wt % are not shown.

### Example 4

### PBSA Acid Neutralization and Effect of Chemical Environment on Hydrogel Integrity

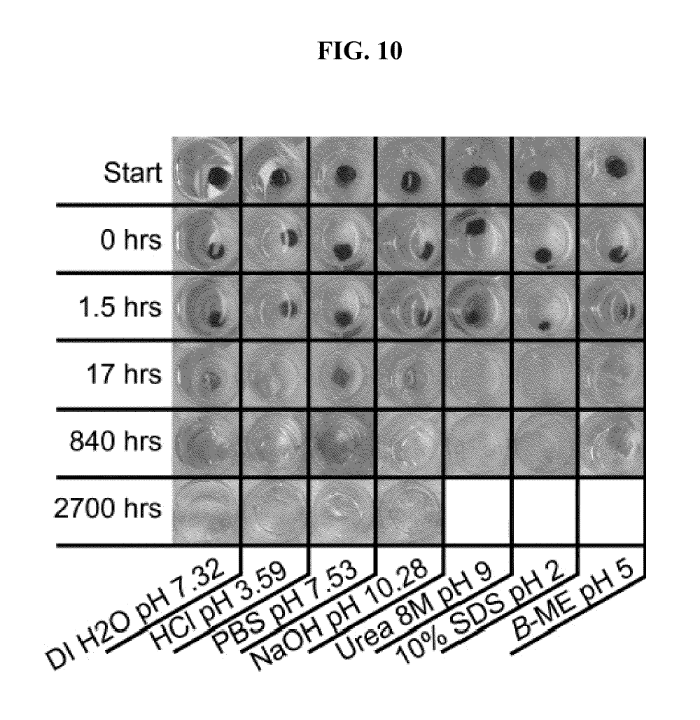

To assess the nature of the interactions within the gel network, 20 wt % PBSA gel samples (pH 3.5) were incubated in different chemical environments (FIG. 10). PBSA hydrogels formed at low pH were stable after acid neutralization to pH 7 by acid leaching in a PBS water bath. Phenol red added to neutralizing PBS buffer colored the PBSA hydrogels red over three days and serve as a visual marker for bulk hydrogel pH. PBSA hydrogels were stable for up to three months in deionized H₂O (pH 7.32), HCl—H2O (pH 3.59), NaOH—H2O (pH 10.28), and in PBS (pH 7.53) indicating a resistance to degradation by acidic or basic conditions. PBSA gels submerged in 8 M Urea or 10% SDS were completely degraded within 17 hours indicating that a primary mechanism of PBSA hydrogel formation is non-covalent and probably driven by hydrophobic interactions. Reduction of intermolecular disulphide bonds by β-Mercaptoethanol (β-ME) solvent resulted in hydrogel swelling.

### Example 5

### In Vivo Biocompatibility Evaluation

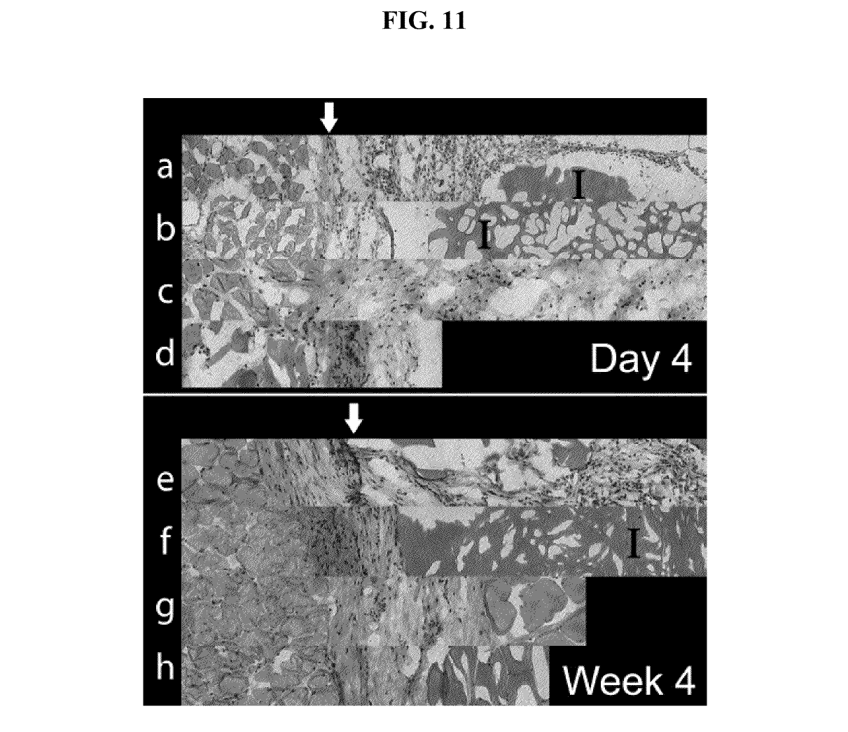

To evaluate the acute and chronic inflammatory potential of the BSA hydrogels in vivo, TBSA and acid-leached PBSA gel disks were implanted subcutaneously. Gross observation of the explants at 4 days and 4 weeks showed that the tissue had grown around the implants. H&E staining of the sections revealed stark differences between the degradation patterns of the PBSA and the TBSA implants in vivo (FIG. 11). Cells infiltrating the PBSA hydrogels were seen at both time points, and correlated with significant gel degradation (FIG. 11, panels a and e). The PBSA hydrogels were noticeably more degraded at 4 weeks than at 4 days although both displayed complete degraded channels traversing the entire length of the implant. In contrast, TBSA hydrogels showed no sign of degradation and were intact at both time points (FIG. 11, panels b and f). A fibrous capsule surrounding the TBSA hydrogels became denser and thicker at 4 weeks relative to 4 days. Control 20 wt % BSA solutions (pH 7.4) injected into subcutaneous pockets resulted in an increased general inflammatory response judged by relative increase in number of cells in the subcutaneous pocket (FIG. 11, panel c) at 4 days when compared to the saline control injection (FIG. 11, panel d). At 4 weeks, the inflammatory response of the BSA injection has decreased significantly (FIG. 11, panel g) and was similar to that of the saline control (FIG. 11, panel h).

### Example 6

### atRA Binds to Both F-Form Albumin and N-Form Albumin Isoforms

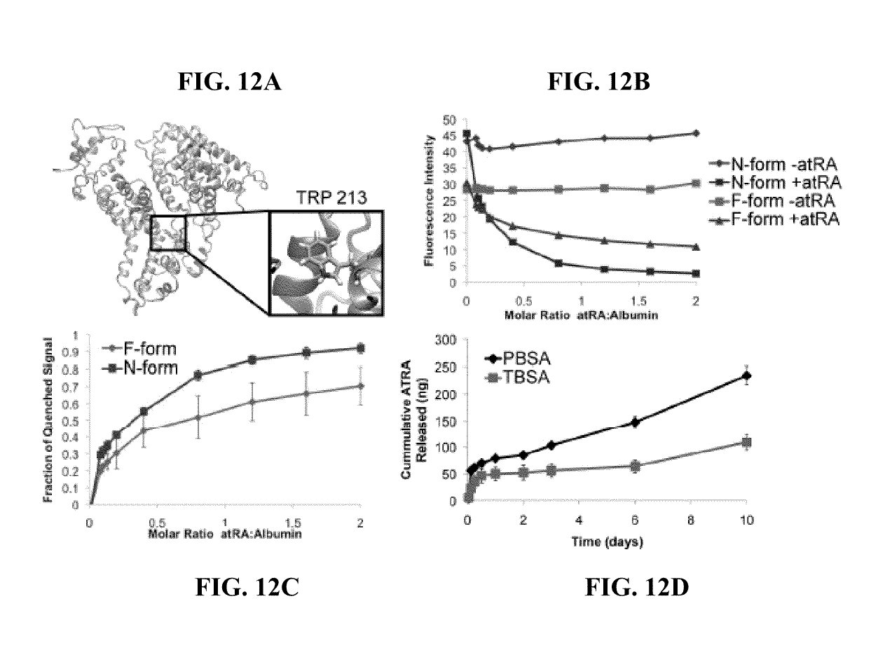

Tryptophan fluorescence at 340 nm changes upon atRA binding to BSA (FIG. 12). One of the two tryptophans in BSA (TRP 213) is located deep within the globin fold in domain II and fluorescence signal from this tryptophan residue is reduced when bound to atRA (FIG. 5A). Without added atRA, N-form albumin exhibits higher initial fluorescence intensity over F-form albumin (FIG. 12B). Upon addition of atRA, the tryptophan fluorescence signal at 340 nm is rapidly quenched for both N-form albumin and F-form albumin. As a fraction of the initial fluorescence signal, N-form albumin exhibits greater fluorescence quenching than F-form albumin (FIG. 12C) that may be due to the altered conformation state of F-form albumin and shifting atRA binding sites to other locations not dominated by the TRP 213 residue. PBSA and TBSA hydrogels loaded with atRA demonstrate a small initial burst release of atRA followed by linear release over 10 days in PBS at 37° C. (FIG. 12D).

### Example 7

### atRA Binding Sites Determined by Molecular Dynamics

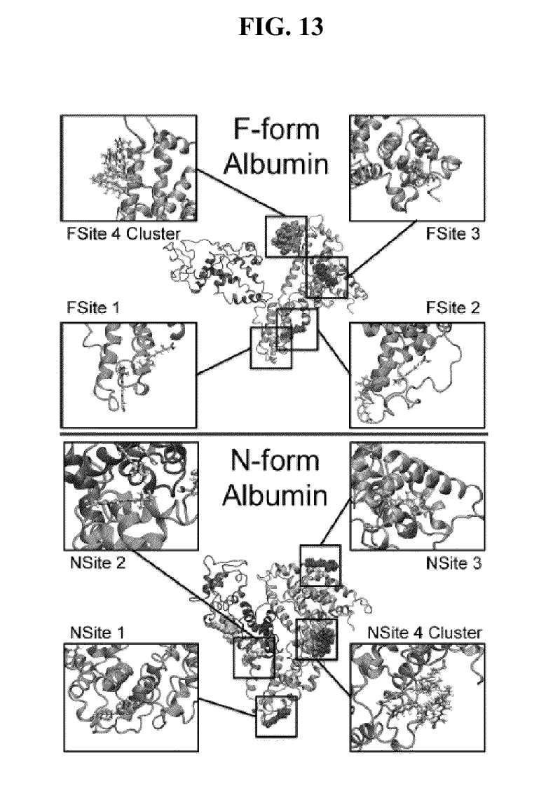

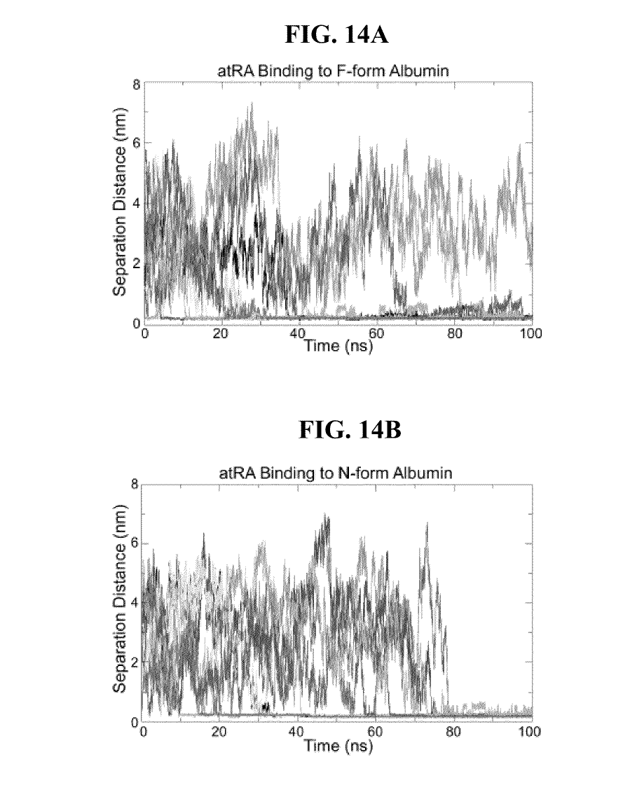

Computational molecular dynamics enables atomic level resolution of the interaction between albumin and atRA during several binding events. Initial random placement of atRA molecules in albumin models for N-form albumin and F-form albumin structures allow for unbiased exploration and binding of atRA molecules to the protein surfaces. Final configurations of atRA on N-form albumin and F-form albumin conformations are represented in FIG. 6. For both structures, there are sites on the protein that were bound to a single atRA molecule (FSite1, FSite2, FSite3, NSite1, NSite2, and NSite3) and areas where clusters of atRA molecules formed an aggregate (FSite4 Cluster, NSite4 Cluster). These clusters were not formed in the water phase but rather formed after an initial atRA molecule became bound to the cluster site. In N-form albumin, atRA molecules were bound to all three domains while F-form albumin had no atRA molecules bound to domain III (FIG. 13). Domain III experiences the greatest degree of denaturation during the N—F transition. atRA binding events on both N-form albumin and F-form albumin were, within the timescale of the simulation, irreversible. When an atRA molecule would approach the surface of the protein near a binding site, it would remain localized to that site as quantified by the successive drop in the separation distance between each atRA molecule and its nearest protein residue surface (FIG. 14A, B). Within 80 ns, 9 out of 10 atRA molecules were bound to the F-form albumin protein surface while all 10 atRA molecules were bound to the N-form albumin surface.

### Example 8

### Potential of Mean Force Calculation for atRA Binding to FSite1 and NSite1

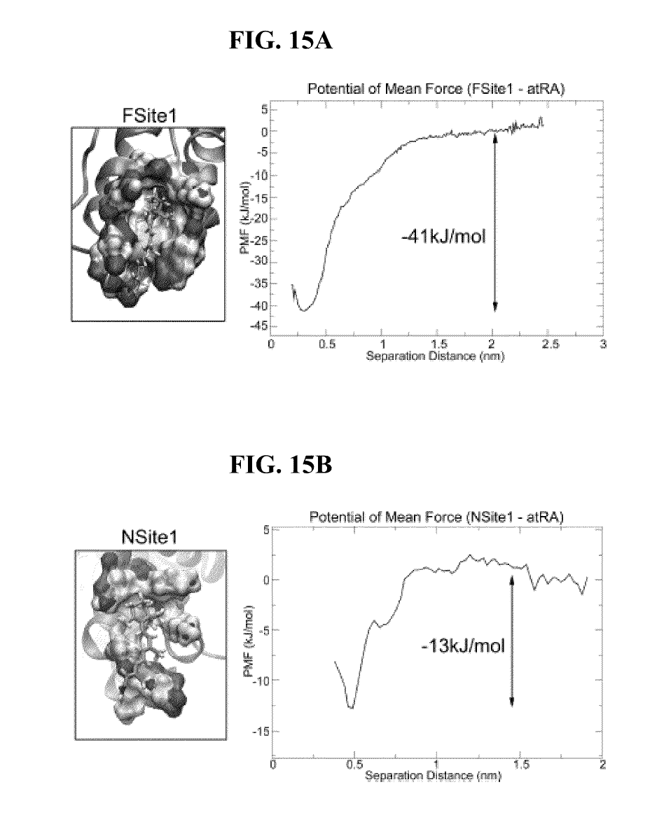

The potential of mean force (PMF) is the free energy of interaction between two molecules. It represents the interaction between the molecules at a fixed separation averaged over all the degrees of freedom of the system, e.g. water molecules, intermolecular and intramolecular interactions and possible rotation and conformations of the protein and the atRA. Therefore, it provides the value for the work required to bring an atRA molecule from the bulk to a distance z from a BSA binding site. The potential of mean-force difference between infinity separation, defined as zero, and that of the minimum represents the free energy of binding. An objective in calculating the PMF for FSite1 and NSite1 was to evaluate whether the F-form albumin conformations locked in the PBSA hydrogels retain atRA binding affinity comparable to N-form albumin. Literature estimates the binding energy for the fluorescence-quenching TRP 213 atRA binding site on N-form albumin are −31.7 kJ/mol (Maiti et al. Int J Biol Macromol. 2006; 38(3-5):197-202; herein incorporated by reference in its entirety). PMF calculations demonstrate that the FSite1 has a binding energy of −41 kJ/mol and the NSite1 has a binding energy of −13 kJ/mol (FIG. 15). The optimal separation distance between the center of mass of atRA and the center of mass on FSite1 is 0.32 nm and with NSite1 it is 0.48 nm. These results demonstrate that the F-form albumin conformation retains a strong binding affinity towards atRA.

### Example 9

### Released atRA Inhibits HASMC Migration

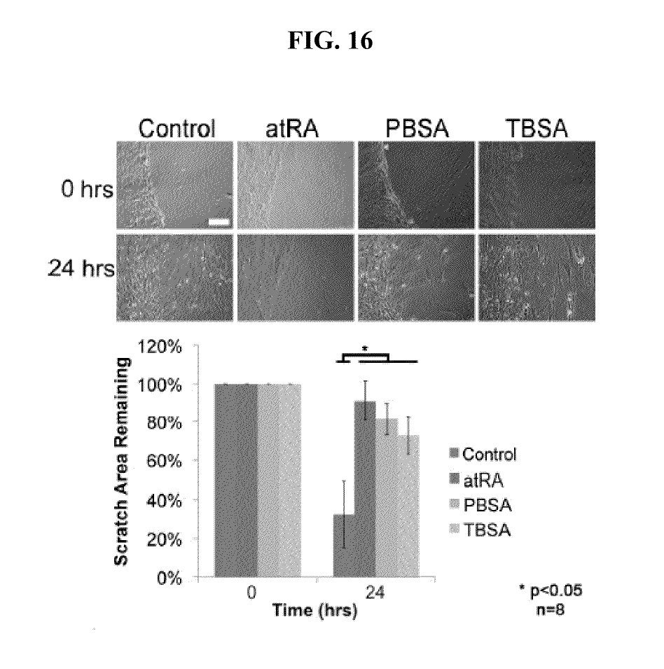

The bioactivity of atRA released from PBSA or TBSA gels was evaluated in a scratch wound assay. After allowing HASMCs to grow to 90% confluence and migration priming in serum-starved media, elution from day 10 of the release study was added to the cell culture. As expected, the migration 450 of positive control cells exposed directly to 25 ng/mL atRA added in the media was inhibited in comparison to the negative control cells (FIG. 16). This result confirms the initial bioactivity of the atRA to inhibit HASMC migration. HASMC migration for cells receiving atRA released from PBSA and TBSA gels was also inhibited and significantly different from the negative control.

### Example 10

### F-Form Crystal Structure for HSA and Other Drug Interactions

HSA and BSA are close homologs (85.5% structural similarity, 75.6% sequence identity match; Chruszcz et al. Bba-Gen Subjects 1830, 5375-5381 (2013); herein incorporated by reference in its entirety) and BSA provides high-level prediction of comparable crystal structure conformations of HSA. Experiments conducted during development of embodiments of the present invention successfully fabricated PHSA hydrogels similar to the PBSA. MD simulations are performed on N-form crystal structures of HSA and rHSA to determine their partially denatured F-form crystal structures. With these F-form crystal structures it is possible to further characterize the drug binding interactions and identify new binding sites for a variety of other therapeutic molecules (Wang et al. Bba-Gen Subjects 1830, 5356-5374; Yamasaki et al. Bba-Gen Subjects 1830, 5435-5443; herein incorporated by reference in their entireties). Further drug-F-form albumin interaction studies include cisplatin, warfarin, phenylbutazone, azapropazone, indomethacin, tolbutamide, iodipamide, iophenoxic acid, furosemide, bucolome, sulfisoxazole, diazepam, diflunisal, ibuprofen, ketoprofen, naproxen, 6-MNA, diclofenac, etodolac, clofibrate, iopanoic acid, probenecid, amitrityline, debrisoquine, digitoxin, propofol, fusidic acid, lidocaine, bicalutamide, camptothecin, 9-amino therapeutic molecules, and PBSA or PHSA systems that confer additional degradation resistance (Yamasaki et al. Bba-Protein Struct M 1295, 147-157; Kragh-Hansen. Mol Pharmacol 34, 160-171 (1988).; Montero et al. J Pharm Pharmacol 38, 925-927 (1986).; Zini et al. Biochem Pharmacol 28, 2661-2665 (1979).; Vallner. J Pharm Sci-Us 66, 447-465 (1977).; Takamura et al. Drug Metab Dispos 33, 596-602 (2005).; Anton. Annals of the New York Academy of Sciences 226, 273-292 (1973).; Verbeeck et al. Biochem Pharmacol 29, 571-576 (1980).; Chuang et al. Bba-Protein Struct M 1434, 18-30 (1999).; Honore & Brodersen. Mol Pharmacol 25, 137-150 (1984).; Setoguchi et al. Biopharm Drug Dispos 34, 125-136 (2013).; Mignot et al. Chirality 8, 271-280 (1996).; Meisner & Neet. Mol Pharmacol 14, 337-346 (1978).; Bhattacharya et al. JBiol Chem 275, 38731-38738 (2000).; Zunszain et al. J Mol Biol 381, 394-406 (2008).; Hein et al. J Struct Biol 171, 353-360 (2010).; Ivanov et al. J Biol Chem 273, 14721-14730 (1998).; Sudlow et al. Mol Pharmacol 12, 1052-1061 (1976).; Kragh-Hansen. Biochem J225, 629-638 (1985).; Sjoholm et al. Mol Pharmacol 16, 767-777 (1979).; herein incorporated by reference in their entireties). Analysis of binding interactions between therapeutic molecules and F-form may also consider the change in degradation rates when exposed to urea and also in physiological conditions (e.g., exposure to proteases or in vivo implantation).

### Example 11

### F-Form Albumin Microparticle Fabrication for Drug Delivery

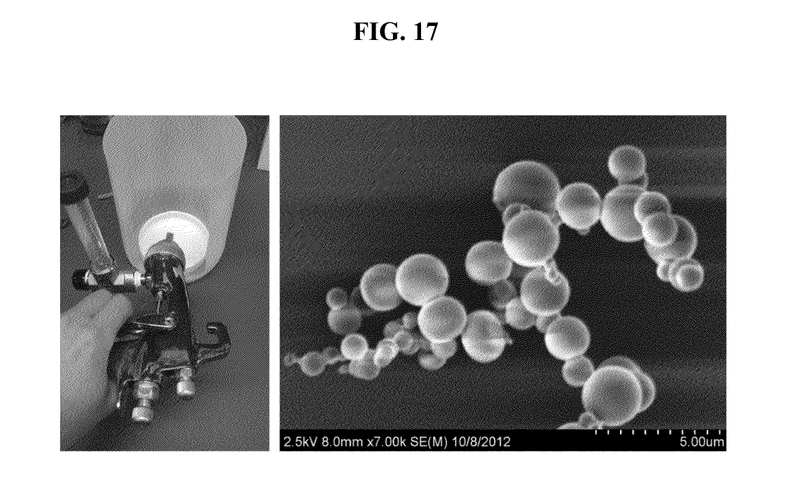

Experiments were conducted during development of embodiments of the present invention to develop a preliminary scalable and inexpensive technique to prepare albumin microparticles that exploit the F-form albumin structures. A simple automotive paint sprayer aerosolizes a solution of albumin at physiological pH 7.4 (FIG. 17, left). The particle spray falls into a bath at pH 3.5 resulting in immediate partial denaturation of the albumin and formation of spherical particles (ranging from 100 nm-2.5 μm in diameter) from the spray (FIG. 17, right). Through this partial denaturation procedure, therapeutic molecules are bound to albumin at different stages, either prior to aerosolization or after gelation. After particle formation, the acid used in the spray bath is neutralized without altering the microparticles.

Therapeutics are either added into the solution of albumin prior to aerosolization or into the low pH bath for loading into the hydrogels. In the former case, therapeutics added to the pre-aerosolized albumin solution bind to N-form binding sites, which may stabilize the albumin during partial degradation to the F-form. Lowering the pH of the bath induces the necessary partial denaturation of the albumin. In the latter case, the therapeutics are stable in a low pH environment and will bind to the F-form albumin as it is sprayed into the bath. In either case, inclusion of therapeutics may further stabilize the PBSA nanoparticle system as observed preliminarily in the bulk PBSA hydrogel observations.

### Sequences

SEQ ID NO: 1 - Bovine Serum Albumin (BSA):
MKWVTFISLLLLFSSAYSRGVFRRDTHKSEIAHRFKDLGEEHFKGLVLIA
FSQYLQQCPFDEHVKLVNELTEFAKTCVADESHAGCEKSLHTLFGDELCK
VASLRETYGDMADCCEKQEPERNECFLSHKDDSPDLPKLKPDPNTLCDEF
KADEKKFWGKYLYEIARRHPYFYAPELLYYANKYNGVFQECCQAEDKGAC
LLPKIETMREKVLASSARQRLRCASIQKFGERALKAWSVARLSQKFPKAE
FVEVTKLVTDLTKVHKECGHGDLLECADDRADLAKYICDNQDTISSKLKE
CCDKPLLEKSHCIAEVEKDAIPENLPPLTADFAEDKDVCKNYQEAKDAFL
GSFLYEYSRRHPEYAVSVLLRLAKEYEATLEECCAKDDPHACYSTVFDKL
KHLVDEPQNLIKQNCDQFEKLGEYGFQNALIVRYTRKVPQVSTPTLVEVS
RSLGKVGTRCCTKPESERMPCTEDYLSLILNRLCVLHEKTPVSEKVTKCC
TESLVNRRPCFSALTPDETYVPKAFDEKLFTFHADICTLPDTEKQIKKQT
ALVELLKHKPKATEEQLKTVMENFVAFVDKCCAADDKEACFAVEGPKLVV
STQTALA
SEQ ID NO: 2 - Human Serum Albumin (HSA):
MKWVTFISLLFLFSSAYSRGVFRRDAHKSEVAHRFKDLGEENFKALVLIA
FAQYLQQCPFEDHVKLVNEVTEFAKTCVADESAENCDKSLHTLFGDKLCT
VATLRETYGEMADCCAKQEPERNECFLQHKDDNPNLPRLVRPEVDVMCTA
FHDNEETFLKKYLYEIARRHPYFYAPELLFFAKRYKAAFTECCQAADKAA
CLLPKLDELRDEGKASSAKQRLKCASLQKFGERAFKAWAVARLSQRFPKA
EFAEVSKLVTDLTKVHTECCHGDLLECADDRADLAKYICENQDSISSKLK
ECCEKPLLEKSHCIAEVENDEMPADLPSLAADFVESKDVCKNYAEAKDVF
LGMFLYEYARRHPDYSVVLLLRLAKTYETTLEKCCAAADPHECYAKVFDE
FKPLVEEPQNLIKQNCELFEQLGEYKFQNALLVRYTKKVPQVSTPTLVEV
SRNLGKNGSKCCKHPEAKRMPCAEDYLSVVLNQLCVLHEKTEVSDRVTKC
CTESLVNRRPCFSALEVDETYVPKEFNAETFTFHADICTLSEKERQIKKQ
TALVELVKHKPKATKEQLKAVMDDFAAFVEKCCKADDKETCFAEEGKKLV
AASQAALGL

All publications and patents in the above specification are herein incorporated by reference. Various modifications and variations of the described method and system of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

1. A composition comprising a hydrogel of partially-denatured albumin.

2. The composition of claim 1, wherein the partially-denatured albumin is electrostatically denatured.

3. (canceled)

4. The composition of claim 3, wherein the partially-denatured albumin is F-form albumin.

5. The composition of claim 1, wherein the partially-denatured albumin in the hydrogel retains drug-binding functionality of undenatured albumin.

6. The composition of claim 5, wherein the partially-denatured albumin in the hydrogel binds all-trans retinoic acid.

7. The composition of claim 1, wherein the albumin has at least 70% sequence identity with SEQ ID NO: 1 or SEQ ID NO: 2.

8. (canceled)

9. The composition of claim 1, wherein the albumin in the hydrogel is substantially not cross-linked.

10. The composition of claim 1, wherein the albumin in the hydrogel is not completely denatured.

11. The composition of claim 1, wherein the hydrogel is non-toxic to humans.

12. The composition of claim 1, further comprising cells or one or more active agents.

13. The composition of claim 12, wherein the cells or one or more active agents are embedded within the hydrogel.

14. The composition of claim 12, wherein the cells or one or more active agents are in a reservoir within the hydrogel.

15. The composition of claim 12, wherein the hydrogel has a diffusion coefficient for the active agent between 1×10⁻⁷ and 1×10⁻⁴ mm²/minute.

16. A device comprising at least one base material, wherein at least a portion of the at least one base material is coated with the composition of claim 1.

17. The device of claim 16, wherein the device is a medical device, a surgical device, or an implantable device.

18. (canceled)

19. The device of claim 16, wherein said composition further comprises an active agent.

20. A drug-delivery device comprising the composition of claim 1 and a therapeutic agent.

21. (canceled)

22. A method of treating or preventing a condition in a subject comprising:
(a) administering the drug-delivery device of claim 20 to the subject, wherein the therapeutic agent is effective in the treatment or prevention of the condition; and
(b) allowing: (i) the therapeutic agent to elute from the hydrogel, and/or (ii) the hydrogel to degrade, thereby releasing the therapeutic agent.

23.-33. (canceled)

34. A hydrogel particle comprising the composition of claim 1.

35. (canceled)

36. A method of producing the hydrogel particle of claim 34, comprising aerosolizing a solution comprising albumin into a solution below pH 5.0.

37.-48. (canceled)
